SEQUENCE LISTING (1 sequence)

SEQ ID NO: 1 - DNA, 13 bp, Artificial Sequence
  Description of Artificial Sequence Synthetic oligonucleotide
tttcttatatggg

(19) United States
(12) Patent Application Publication
(10) Pub. No.: US 2023/0408514 A1
(43) Pub. Date: Dec. 21, 2023

(54) SINGLE-CELL COMBINATORIAL INDEXED CYTOMETRY SEQUENCING

(71) Applicants: Chan Zuckerberg Biohub, Inc., San Francisco, CA (US); The Regents of the University of California, Oakland, CA (US)

(72) Inventors: Byungjin Hwang, Oakland, CA (US); David Sungjin Lee, Oakland, CA (US); Chun Ye, Oakland, CA (US)

(21) Appl. No.: 17/911,509

(22) PCT Filed: Mar. 18, 2021
(86) PCT No.: PCT/US2021/023039

§ 371 Date: Sep. 14, 2022

Related U.S. Application Data
(60) Provisional application No. 62/991,529, filed on Mar. 18, 2020.

(51) Int. Cl. G01N 33/569; G01N 33/53; C12Q 1/6804

(52) U.S. Cl. CPC G01N 33/56972; G01N 33/5308; C12Q 1/6804

(57) ABSTRACT

The development of DNA-barcoded antibodies to tag cell-surface molecules has enabled the use of droplet-based single cell sequencing (dsc-seq) to profile the surface proteomes of cells. Compared to flow and mass cytometry, the major limitation of current dsc-seq-based workflows is the high cost associated with profiling each cell, thus precluding its use in applications where millions of cells are required. Here, we introduce SCITO-seq, a new workflow that combines combinatorial indexing and commercially available dsc-seq to enable cost-effective cell surface proteomic profiling of greater than 10⁵ cells per microfluidic reaction. We demonstrate SCITO-seq's feasibility and scalability by profiling mixed species cell lines and mixed human T and B lymphocytes. To further demonstrate its applicability, we used SCITO-seq to obtain cellular composition estimates in peripheral blood mononuclear cells across two donors that are reproducible and comparable to those obtained by mass cytometry. SCITO-seq can be extended to include simultaneous profiling of additional modalities such as transcripts and accessible chromatin or tracking of experimental perturbations such as genome edits or extracellular stimuli.

### CROSS-REFERENCE TO RELATED APPLICATIONS

This application is a national phase application of PCT Application No. PCT/US2021/023039, filed Mar. 18, 2021, which claims benefit of U.S. provisional application No. 62/991,529, filed Mar. 18, 2020, the entire content of which is incorporated herein by reference.

### BACKGROUND

The use of DNA to barcode physical compartments and tag intracellular and cell-surface molecules has enabled the use of sequencing to efficiently profile the molecular properties of thousands of cells simultaneously. While initially applied to measuring the abundances of RNA^{1,2} and identifying regions of accessible DNA³, recent developments in DNA-tagged antibodies have created new opportunities to use sequencing to measure the abundances of cell surface proteins^{4,5} and intracellular proteins⁶.

Sequencing DNA-tagged antibodies is particularly useful for profiling cells whose identity and function have long been determined by cell surface proteins (e.g. immune cells) and has several advantages over flow and mass cytometry. First, the number of cell surface proteins that can be measured by DNA-tagged antibodies is exponential to the number of bases in the tag. In theory, all cell surface proteins with available antibodies can be targeted and in practice, panels targeting hundreds of proteins are now commercially available^{4,7}. This contrasts with cytometry where the number of proteins targeted is limited by the overlap in the emission spectrums of fluorophores (flow: 4-48) or the number of unique masses of metal isotopes that can be chelated by commercial polymers (CYTOF: ^{˜}50)^{8,9}. Second, sequencing-based proteomics can readily read out all antibody tagging sequences with one reaction instead of subsequent rounds of signal separation and detection, significantly reducing the time and sample input for profiling large panels and obviates the need for fixation. Third, additional molecules can be profiled within the same cell enabling multimodal profiling of cell surface proteins along with the immune repertoire, transcriptome⁴, and potentially the epigenome. Finally, sequencing is amenable to encoding orthogonal experimental information using additional DNA barcodes (either inline or distributed) creating opportunities for large-scale multiplexed screens that barcode cells using natural variation¹⁰, synthetic sequences^{11,12}, or sgRNAs^{13,14}.

### BRIEF DESCRIPTION OF THE INVENTION

In one aspect provided is an assay method comprising tagging cell surface molecules of cells with DNA-barcoded antibodies and using droplet-based single cell sequencing to determine protein expression profiles of the cells wherein at least 30% of droplets comprise multiple cells and the protein expression profiles for multiple cells simultaneously encapsulated in a single drops are resolved by the combinatorial index of barcodes.

In one aspect provided is an assay method comprising (a) providing a plurality of vessels, each vessel comprising i-a) a plurality of cells from a population, each cell comprising a plurality of cell surface proteins, and ii-a) a panel of staining constructs, wherein each staining construct comprises a handle-tagged antibody and a pool oligonucleotide, wherein each handle-tagged antibody comprises iii-a) an antibody specific for a cell surface protein in (i-a), and iv-a) a handle oligonucleotide attached to the antibody, wherein the handle oligonucleotide comprises a handle sequence that identifies the specificity of the antibody to which it is attached; and each pool oligonucleotide comprises at least the following nucleotide segments: v-a) a handle complement segment complementary to, and annealed to, the handle oligonucleotide, vi-a) a capture complement segment, vii-a) an antibody barcode complement segment having a sequence that identifies the binding specificity of the antibody in (iii-a) and thereby identifies the handle oligonucleotide in (iv-a), and viii-a) a pool barcode complement segment, wherein (vii-a) and (viii-a) are positioned between (v-a) and (vi-a), wherein in each vessel, the staining constructs in the vessel have the same pool barcode complement segments, wherein in at least some vessels at least one staining construct is to a cell surface protein in (i-a); (b) optionally combining the contents of all or some of said plurality of vessels, (c) loading individual stained cells or combinations of individual stained cells into compartments, wherein each stained cell comprises one or more staining constructs bound to a cell surface protein of the cell wherein at least some compartments comprise one or more stained cells and a plurality of droplet oligonucleotides wherein each droplet oligonucleotide comprises a droplet bar code and a capture segment wherein the droplet oligonucleotides in a compartment have the same droplet barcode and droplet oligonucleotides in different compartments have different barcodes wherein the capture segment is complementary to and anneals to the capture complement segment of the pool oligonucleotide; (d) producing sequence fragment structures corresponding to the capture constructs, each sequence fragment structure comprising a droplet barcode, a pool barcode and an antibody barcode whereby a plurality of sequence fragment structures are produced (e) sequencing at least some of the plurality of sequence fragment structures to determine the sequences of the droplet barcode, the pool barcode and the antibody barcode of individual sequence fragment structures; (f) determining from the sequencing in (e) distribution of cell surface proteins on individual cells. The pool barcode and antibody barcode are a compound barcode.

In an approach in step (c) at least some of the compartments have two or more cells loaded therein, and cell surface protein expression profiles of said two or more cells are determined. In some cases at least 30% of the compartments containing cells comprise two or more cells. In some cases the cells in the plurality of vessels in (a) comprise a cell population and a composition or expression of cell surface proteins in the population is determined. In some cases the compartments are droplets or wells. In some cases droplet oligonucleotides (capture oligonucleotides) are attached to beads.

In an aspect provided is a nucleic acid capture complex comprising a handle oligonucleotide, a pool oligonucleotide, and a droplet oligonucleotide. In an aspect provided is a kit comprising two or more of (i) a plurality of handle-tagged antibodies comprising different handle sequences and antibodies with different binding specificities, wherein there is a correlation between each handle sequence and each antibody specificity; (ii) a plurality of pool oligonucleotides with different handle complement sequences, wherein said handle complement sequences are complementary to and can anneal to the handle sequences in (i); and (iii) a plurality of droplet oligonucleotides configured to combine with pool oligonucleotides.

### DESCRIPTION OF THE DRAWINGS

FIG. 1 provides diagrams to assist the reader and illustrates elements of one of many embodiments of an aspect of the invention. The illustration is not intended to limit the invention. A=Handle-Tagged Antibody; B=Pool Oligonucleotide (also called a “Splint Oligo,” “Ab-Pool Oligo” or “Secondary Oligo”); C=Droplet Oligonucleotide; A+B=“Staining Construct”; A+B+C=“Capture Construct.” In FIG. 1 (upper panel), the mAb is shown attached at the 3′ terminus of the Handle. It will be recognized that the mAb can be attached at other sites on the Handle sequence. For example, in FIG. 6A the Handle is attached to the antibody at the 5′ terminus. The position of attachment may be selected to avoid steric interference with enzymes, cell surface proteins (CSPs), other polynucleotides, and other elements.

FIG. 2: Design of SCITO-seq and mixed-species proof-of-concept experiment. (a) SCITO-seq workflow. Antibodies are first each conjugated with a unique antibody barcode and hybridized with an oligo containing the compound antibody and pool barcodes (Ab+Pool BC). Cells are split and stained with specific antibodies per pool. Stained cells are pooled and loaded for droplet-based sequencing at high concentrations. Cells are resolved from the resulting data using the combinatorial index of Ab+Pool BC and droplet barcodes. (b) A detailed structure of the SCITO-seq fragment produced. The primary universal oligo is an antibody specific hybridization Handle. The Pool Oligo includes the reverse complement sequence to the Handle followed by a TruSeq adaptor, the compound Ab+Pool barcode, and the 10×3′v3 feature barcode sequence (FBC). The Ab+Pool barcode and the droplet barcode (DBC) forms a combinatorial index unique to each cell. (c) Cost savings and collision rate analysis. As the number of pools increases, total library and DNA-barcoded antibody construction costs drop (left) while the number of cells recovered increase (right). Number of cells recovered as a function of the number of pools at three commonly accepted collision rates (1%, 5% and 10%). (d) Mixed species (HeLa and 4T1) proof-of-concept experiment. HeLa and 4T1 cells are mixed and stained in five separate pools at a ratio of 1:1 with SCITO-seq antibodies barcoded with pool-specific barcodes. Scatter (left) and density (right) plots of (e) 38,504 unresolved cell-containing droplets (CCD) and (f) 52,714 resolved cells at a loading concentration of 1×10⁵ cells. Merged antibody derived tag (ADT) counts are generated by summing all counts for each antibody across pools simulating standard workflows. Resolved data is obtained after assigning cells based on the combination of Ab+Pool and DBC barcodes.

FIG. 3: Demonstration of SCITO-seq in human donor experiment with significant increase in throughput of profiling proteins. (a) Schematic of human mixing experiment where different ratios of T and B cells (5:1 and 1:3) were pooled prior to splitting and indexing with five pools of CD4 and CD20 antibodies. Cell type donors are indicated by color while shapes indicate donors. Scatter plot and density plots of (b) unresolved and (c) resolved cells for loading concentrations of 1×10⁵ (left) and 2×10⁵ (right) cells. (d) Expected (x-axis) versus observed (y-axis) frequencies of co-occurrences between antibody and pool barcodes for loading concentrations of 1×10⁵ (left) and 2×10⁵ (right) cells. Expected frequencies were calculated based on the frequencies of barcodes in singlets. (e) Distribution of the normalized UMI counts for each antibody in cells resolved from singlets and multiplets per donor. Distribution of the antibodies in multiplets shows expected prior mixture proportions and overlaps with the corresponding distribution in singlets.

FIG. 4: Large-scale PBMC profiling of healthy controls using antibody counts. (a) UMAP projection of single cell expression based on antibody counts showing major lineage markers (Top row) for 200K loading. Resolved UMAP based on antibody counts (b) UMAP comparing the singlets and multiplets (c). Correlations of cell type proportions between singlets and multiplets within donor and across donor (d). CyTOF and SCITO-seq comparison of estimated cell type proportions per donor (e). Downsampling experiment with Adjusted Rand Index measurement and corresponding UMAP based on antibody counts (f). Total cost estimates (purple) including library prep, antibody prep and sequencing cost (g).

FIGS. 2, 3, and 4 are found in color in Hwang et al., SCITO-seq: single-cell combinatorial indexed cytometry sequencing” bioRxiv 2020.03.27.012633; doi: https://doi.org/10.1101/2020.03.27.012633.

FIG. 5: Extending SCITO-seq for compatibility with 60-plex custom and 165-plex commerical antibody panels. (a) UMAP projection of 175,930 resolved PBMCs using a panel of 60-plex antibodies colored by leiden clusters and (b) key lineage markers. Subscripts/prefixes stands for: c:conventional, nc:non-conventional, act:activated, gd:gamma-delta. (c) UMAP projection of 175,000 resolved PBMCs using a panel of 165-plex TotalSeq-C antibodies (TSC 165-plex) colored by leiden clusters and (d) key lineage markers. (e) Distributions of UMIs for multiplicities of encapsulation (MOE) ranging from 1 to 10 cells per droplet for 60-plex (left) and TSC 165-plex (right) experiments. MOE is estimated by Ab+PBC counts for each CCD. (f) Correlation plots for 60-plex (left) and TSC 165-plex (right) experiments comparing estimated (x-axis) and expected MOEs (y-axis). Ten points are shown from MOE of 1 to 10 and colors matched to panel (e). (g) UMAP projection showing the identification of plasmacytoid dendritic cells by CD303. (h) Schematic of sample multiplexed SCITO-seq where different samples are hashed with different pool barcodes. Droplets containing cells from different individuals can be resolved into separate cells. (i) Correlations of the cell composition estimates using the 60-plex (x-axis) versus TSC 165-plex (y-axis) experiments for major cell lineages (T an NK cell (left), B cell (middle), Myeloid cells (right)) for the same 10 donors represented in each pooled experiment.

FIG. 6: Combining SCITO-seq and scifi-RNA-seq for simultaneous profiling of transcripts and surface proteins. (a) Schematic of the SCITO-seq and scifi-RNA-seq coassay. Hγ761 bridized SCITO-seq antibodies are used to stain cells in different pools. Cells are washed with buffer then fixed and permeabilized with methanol. Transcripts undergo in-situ reverse transcription (RT) with pool specific RT primers (well barcode encoded as WBC). RNA and ADT molecules are then captured with RNA- and ADT-specific bridge oligos and ligated to DBCs in-emulsion. Ridgeplots of pool-specific expression from a mixture of cell lines 766 for the (b) RNA library and (c) ADT library. (d) UMAP projection generated from ADT data colored by normalized ADT counts with sample annotations from known markers. (e) Barnyard plot showing expected staining of human anti-CD29 (x-axis) and mouse anti-CD29 (y-axis) antibodies on HeLa cells and 4T1 cells respectively. Other cell lines are negative for both antibodies as expected. (f) UMAP projection by ADT markers (top) and corresponding cell line RNA gene scores using Scanpy's score genes function (bottom). (g) Heatmap of the correlation of RNA (y-axis) and ADT markers (x axis), RNA marker genes are mapped onto cell-type specific ADT clusters for all 5 cell lines. For exam773 ple, 4T1 RNA vs 4T1 ADT calculates how well RNA genes in 4T1 predict well on their respective ADT clusters. The scaled values are standardized z-score scale. In FIG. 6, the Droplet Bar Code is denoted “CBC.” “X” denotes a transcription block (e.g., inverted dT).

### DETAILED DESCRIPTION

### 1. Definitions, Abbreviations, and Terminology

As used herein, “antibody” means an immunoglobulin molecule of any useful isotype (e.g., IgM, IgG, IgG1, IgG2, IgG3 and IgG4); chimeric, humanized and human antibodies, antibody fragments and engineered variants, including, without limitation Fab, Fab′, F(abe)2, F(ab1)2 scFv, dsFv, ds-scFv, dimers, single chain antibodies (scAb), minibodies (engineered antibody constructs comprised of the variable heavy (VH) and variable light (VL) chain domains of a native antibody fused to the hinge region and to the CH3 domain of the immunoglobulin molecule); nanobodies, diabodies (comprising two Fv domains connected by short peptide linkers), and multimers thereof; heteroconjugate antibodies (e.g., bispecific antibodies and bispecific antibody fragments), and other forms that specifically bind to a target polypeptide. “Antibodies” are a type of “affinity reagent” that also includes aptamers, affimers, knottins and the like.

As used herein, the term “monoclonal antibody” has its normal meaning in the art and is an antibody from a population of identical antibodies, including a clonal population produced by cells or a population produced by other means.

As used herein, the term “complementary” refers to Watson-Crick base pairing between nucleotides units of two single stranded nucleic acid molecules or two portions of the same nucleic acid molecule. Complementary sequences or segments can be “exactly complementary” (two nucleic acid segments with 100% complementarity, e.g., the sequence of one segment is the reverse complement of the sequence of the other segment) or “substantially complementary” (two nucleic acid segments with less than 100% complementarity and at least about 80%, at least about 85%, at least about 90%, or at least about 95% complementary). Percent complementarity refers to the percentage of bases of a first nucleic acid segment that can form base pairs with a second nucleic acid segment. Polynucleotides or segments with substantially complementary sequences can anneal to each other under assay conditions to form a double stranded segment. It will be appreciated that a first sequence that can anneal to a second sequence to generate a double-stranded molecule can be referred to as a sequence that is the complement of the second sequence, or, equivalently, the “reverse complement.”

As used herein, two nucleic acid segments that are complementary to each other, or have sequences complementary to each other, or have the relationship in which a first segment has a sequence that is “the complement of” a sequence of a second segment.

As used herein, the terms “anneal” and “hybridize” are used interchangeably to refer to two complementary single stranded nucleic acid segments that base-pair to form a double-stranded segment

As used herein, the term “construct” refers to two or more nucleic acid molecules that are associated by base pairing between a subsequence or segment of a first nucleic acid molecule and a complementary subsequence or segment of a second nucleic acid molecule. Reference to a “Construct” does not include a single, fully double stranded, polynucleotide.

As used herein the term “segment” used in reference to a polynucleotide refers to a defined portion or subsequence of the polynucleotide comprising a plurality of contiguous nucleotides. Typically a segment has 5 to 100 contiguous bases.

As used herein, the terms “oligonucleotide” and “oligo” are used interchangeably and, unless otherwise indicated or clear from context, refer to a single stranded nucleic acid less than 500 bases in length. In some cases, as will be apparent from context, a segment is referred to as an “oligonucleotide” sequence (e.g., “the capture complement is an oligonucleotide sequence contained in a Pool Oligonucleotide”).

As used herein, the terms “nucleic acid” and “polynucleotide” are used interchangeably and usually refer to a single or double-stranded DNA polymer. However, methods and compounds described herein may be carried out using oligonucleotides and Constructs that comprise RNA, DNA/RNA chimeras, and synthetic analogs of DNA or RNA containing non-naturally occurring nucleobase analogs, or analogs of (deoxy)ribose or phosphate or, in the case of DNA, contain uracil in place of thymidine, which are also referred to as nucleic acids or polynucleotides.

As used herein, the term “barcode” or “BC” refers to a short (typically less than 50 bases, often less than 30 bases) nucleic acid sequence that identifies a property of a polynucleotide. For example, in some cases polynucleotides with the same barcode have a common origin, e.g., are from the same vessel or compartment. In various places in this disclosure there is reference, for clarity, to a barcode sequence and a barcode sequence complement. It will be recognized that in a double-stranded polynucleotide the sequence in both strands is informative and can serve as a barcode.

As used herein, the term “vessel” refers to a container in which a solution containing cells, oligonucleotides, and/or constructs can be pooled (combined). Antibody binding and nucleic acid hybridization may occur in a vessel. The term “vessel” does not imply a particular structure or material. Examples of vessels include tubes, wells, and microfluidic chambers.

As used herein, the term “compartment” refers to a structure that can contain one or more cells and one or more nucleic acid Constructs. Examples of compartments include droplets, capsules, wells, microwells, microfluidic chambers, and other containers.

As used herein, “bead” may refer to (but is not limited to) beads of the type used in droplet-based single cell sequencing technologies (inDrop, Drop-seq, and 10X Genomics) which carry or are attached to polynucleotides. Bead technology is well known in the art. Wang et al., 2020, “Dissolvable Polyacrylamide Beads for High-Throughput Droplet DNA Barcoding” Advanced Science 7:8, and references cited therein; Klein et al. Cell 2015, 161, 1187; Macosko et al., Cell 2015, 161, 1202; Lan et al Nat. Biotechnol. 2017, 35, 640; Lareau et al. Nat. Biotechnol. 2019, 37, 916; Stoeckius et al. Nat. Methods 2017, 14, 865; Peterson et al. Nat. Biotechnol. 2017, 35, 936; Zheng et al., Nat. Commun. 2017, 8, 14049.

As used herein, a compartment is “occupied” if it contains at least one cell (i.e., is not empty).

Abbreviations: BC—bar code; CSP—cell surface protein; Ab—antibody; mAb—monoclonal antibody; HTA—Handle-Tagged antibody; HCL—high-concentration loading; UMI—unique molecular identifier.

### 2. Introduction

A major limitation in sequencing-based single-cell proteomics^{4,7} is the high cost associated with profiling each cell, thus precluding its use across population cohorts or large-scale screens where millions of cells would need to be profiled. Like other single-cell sequencing assays, total cost per cell for proteomic sequencing is divided between cost associated with library construction and the cost for sequencing the library. Because the number of protein molecules per cell is 2-6 orders of magnitude higher than RNA¹⁵ and the use of targeting antibodies limits the number of features measured per cell, methods that use tagged antibodies for single cell protein analysis likely yield more information content per read per cell than RNA. However, the costs associated with standard microfluidics based single-cell library construction¹⁶ and conjugation of modified DNA sequences to antibodies⁴ are high. Thus, for single-cell proteomic sequencing to be a compelling strategy for high dimensional phenotyping of millions of cells, there is a major need to develop a workflow that minimizes library and antibody preparation costs.

We describe a simple two round SCI experimental workflow, SCITO-seq, which combinatorically indexes single cells using DNA-tagged antibodies⁴ and microfluidic droplets to enable cost-effective profiling of cell-surface proteins scalable to 10⁵-10⁶ cells (FIG. 2a). First, each antibody is conjugated with an antibody-specific amine modified oligo sequence (antibody Handle, 20 bp) that enables pooled hybridization to minimize the costs associated with generating multiple pools of DNA-tagged antibodies. Second, titrated antibodies are pooled and aliquoted before the addition of an oligo pooll (splint oligos) containing compound barcodes for each antibody and pool combination (Ab+PBC). The splint oligos share common sequences for hybridization with antibody-bound oligos (Ab Handle) and a handle for hybridization with bead-bound sequences within each droplet—for example, the feature barcode sequence (Capture Sequence 1 in the 10X 3′ V3 kit) (FIG. 2b). The design of the antibody and bead hybridization sequences can each be customized for compatibility to commercial antibody conjugation and droplet bead chemistries. Third, cells are separated into pools and stained with pool-specific antibodies. Fourth, the stained cells are pooled and loaded at concentrations tunable to the targeted collision rate followed by processing using a commercially available dsc-seq platform to generate a sequencing library incorporating unique molecular identifiers (UMI) and DBCs. Finally, after sequencing only the antibody derived tags (ADTs), the surface protein expression profiles of multiple or simultaneously encapsulated cells within a droplet (multiplets) within a droplet can be resolved by using the combinatorial index of Ab+PBC and DBC.

Our approach is based, in part, on the discovery that the large number of droplets produced by microfluidic workflows (^{˜}10⁵ for 10X Genomics¹⁶) can be used as a second round of physical compartments for single-cell combinatorial indexing (SCI)¹⁷⁻²⁰ resulting in a simple and cost-effective two-step procedure for library construction.

Disclosed herein is a strategy using universal conjugation followed by pooled hybridization to generate large panels of DNA tagged antibodies referred to as “Handle-Tagged antibodies” or “HTA”. Handle-Tagged antibodies are then used to stain cells in individual pools prior to high-concentration loading using commercially available microfluidics devices and methods. Using the current invention, an Antibody Barcode or Handle can be used to identify a cell-surface protein displayed on a cell. Protein expression profiles for multiple (two or more) cells simultaneously encapsulated in a single drop is resolved by the combinatorial index of pool and droplet barcodes. The high concentration loading of stained cells and targeted sequencing reduce the library construction and sequencing costs per cell respectively compared to other single cell sequencing workflows. We demonstrate the feasibility and scalability of SCITO-seq in mixed species and mixed individual experiments profiling 10⁵ cells per microfluidic reaction, a 4-fold increase in throughput compared to standard workflows at the same collision rates. We further illustrate an application of SCITO-seq by profiling 5×10⁴-10⁵ peripheral blood mononuclear cells using a panel of 28 antibodies in one microfluidic reaction from two healthy donors and benchmark the results with mass cytometry (CyTOF). Finally, we demonstrate that targeted sequencing using SCITO-seq can recover the same cell clusters at lower sequencing depths per cell. SCITO-seq can be integrated with existing workflows for multimodal profiling of transcripts²² and accessible chromatin²¹ and can be a compelling platform for obtaining rich phenotyping data from high-throughput screens of genetic and extracellular perturbations.

### 3. Handle, Antibody, and Handle-Tagged Antibody

Antibodies (or other affinity reagents) used in the invention are attached or conjugated to an oligonucleotide referred to as a “Handle” or “Handle sequence.” The antibody and attached Handle are referred to herein as a “Handle-Tagged Antibody” or “HTA.” Other terms that may be used to describe the antibody-handle complex include “tagged-antibody,” “barcoded antibody,” and “DNA-tagged antibody.” In one approach, each different Handle corresponds to a specific monoclonal antibody or binding specificity.

Handle

The Handle is long enough to form a stable complex with the Handle Complement, described below, under assay conditions. Generally, the Handle is at least 10 bases in length, more often 15 bases in length and often 20 bases in length or longer. For example and not limitation, the length of the Handle can be 10-100 bases, 15-50 bases, or 15 to 25 bases.

Antibodies

The antibody portion of the Handle-Tagged Antibody is typically a monoclonal antibody such as a monoclonal antibody specific for a cell-surface protein (“CSP”). In some embodiments, an antibody specific for a cell-surface protein binds an epitope on the extracellular portion of a cell-surface transmembrane protein. In some embodiments, an antibody specific for a cell-surface protein binds an epitope on a peripheral membrane protein.

It will be recognized that there are a large number of different cell surface proteins. A CSP is generally a naturally occurring protein expressed by a defined, or definable, cell type or types. That is, knowledge of the CSPs expressed by a cell provide information about the cell properties, including type, species, developmental or metabolic state and the like. Any sort of cell can be characterized using the methods of the invention, including cells from an animal, such as a primate (e.g., such as a human), plant, or fungus, and microorganisms.

In certain embodiments the CSP is expressed by and displayed on an immune system cell, such as a lymphocyte, neutrophil, eosinophil, basophil or monocyte. Useful CSPs displayed on immune cells include proteins referred to by cluster of differentiation (CD) designations assigned by HLDA (Human Leukocyte Differentiation Antigens) Workshops. See for example, Beare et al., 2008, “The CD system of leukocyte surface molecules: Monoclonal antibodies to human cell-surface antigens.” Curr. Protoc. Immunol. 80:A.4A.1-A.4A.73, incorporated herein by reference. Exemplary CD proteins are listed in TABLE 1 along with exemplary monoclonal antibodies.

TABLE 1

| CD Designation | Exemplary cell type | Exemplary mAb |
|---|---|---|
| CD45 | Leukocytes | HI30 |
| CD33 | Myeloid cell | WM53 |
| CD3 | T cell | UCHT1 |
| CD19 | B cell | HIB19 |
| CD117 | Hematopoietic stem cell | 104D2 |
| CD11b | Monocytes | IRCF44 |
| CD4 | CD4⁺ T cell | RPA-T4 |
| CD8 | CD8⁺ T cell | RPA-T8 |
| CD11c | Monocytes | BU15 |
| CD14 | CD14⁺ Monocyte | RMO52 |
| CD127 | CD4⁺ T cell | A019D5 |
| FceR1 | Dendritic cell | AER-37 |
| CD123 | Plasmacytoid dendritic ell | 6H6 |
| gdTCR | T cell | 11F2 |
| CD45RA | Naïve T cell | HI100 |
| TIM3 | T cell | F38-2E2 |
| PD-L1 | T cell | 29E.2A3 |
| CD27 | T cell | L128 |
| CD45RO | Memory T cell | UCHL1 |
| CCR7 | T cell | G043H7 |
| CD25 | Regulatory T cell | 2A3 |
| TCR_Va24_Ja18 | Invariant NKT cell | 6B11 |
| CD38 | B cell | HIT2 |
| HLA_DR | Antigen presenting cell (B-cell, | L243 |
| | Macrophage, Dendritic cell) | |
| PD-1 | Activated T cell | EH12.2H7 |
| CD56 | Natural Killer Cell | NCAM16.2 |
| CD235 | Erythrocyte | HIR2 |
| CD61 | Platelet | VI-PL2 |

In certain embodiments the CSP is expressed by and displayed on a cell other than an immune system cell. See for example, Bausch-Fluck et al., 2015, “A Mass Spectrometric-Derived Cell Surface Protein Atlas. PLoS ONE 10(4): e0121314. Bausch-Fluck et al., 2015, “The in silico human surfaceome” Proceedings of the National Academy of Sciences November 2018, 115 (46) E10988-E10997; Fonseca et al., 2016, “Bioinformatics Analysis of the Human Surfaceome Reveals New Targets for a Variety of Tumor Types,” International Journal of Genomics Volume 2016, Article ID 8346198. Suitable monoclonal antibodies are described in public databases (e.g., Genbank, NCBI, EMBL, AbMiner, Antibody Central, European Collection of Cell Cultures, The Hybridoma Databank, Monoclonal Antibody Index). New monoclonal antibodies against any specific antigen can be prepared by art-known methods.

In some embodiments the invention is used to detect or quantitate proteins other than cell surface proteins (e.g., cytoplasmic proteins).

Association of Handle and Antibody.

Generally each different antibody is associated with a unique Handle sequence so that determining a Handle sequence identifies properties of the antibody. In general each antibody used in an assay has a different CSP specificity (e.g., anti-CD2, anti-CD17) which is identified by the Handle sequence. In some embodiments two different antibodies recognize the same CSP but, for example, bind to different epitopes and/or have different isotypes. In some embodiments two different antibodies linked to different Handle sequences recognize the same CSP but in different configurations (e.g., distinguishing dimers from monomers). In some embodiments two antibodies with different specificities are tagged with the same Handle sequence, if there is no need to distinguish the corresponding CSPs.

Attachment of the Handle to the Antibody to Form the Handle-Tagged Antibody.

Methods for attaching the Handle oligonucleotide and the antibody to produce the Handle-Tagged Antibody are known in the art. See, e.g., Stoeckius et al., 2018, Genome Biol. 19:224; Peterson et al., 2017, Multiplexed quantification of proteins and transcripts in single cells Nature Biotechnology 35:936-939. In one approach, the Handle oligonucleotide is an amine modified oligonucleotide conjugated to the antibody or a polypeptide constituent thereof. The Handle can be attached to the antibody at its 5-prime end or its 3′ end depending on downstream steps.

### 4. Pool Oligonucleotide/Splint Oligonucleotide

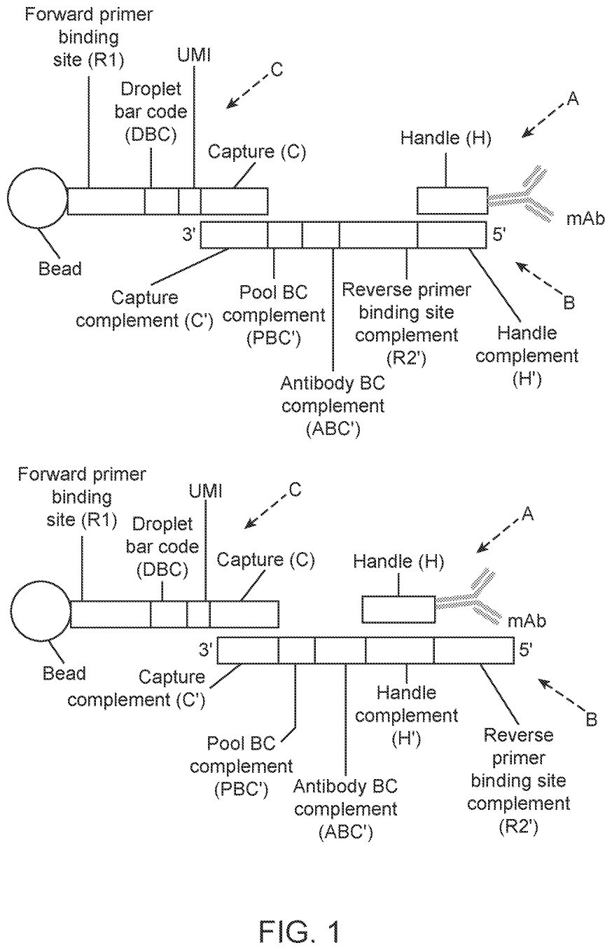

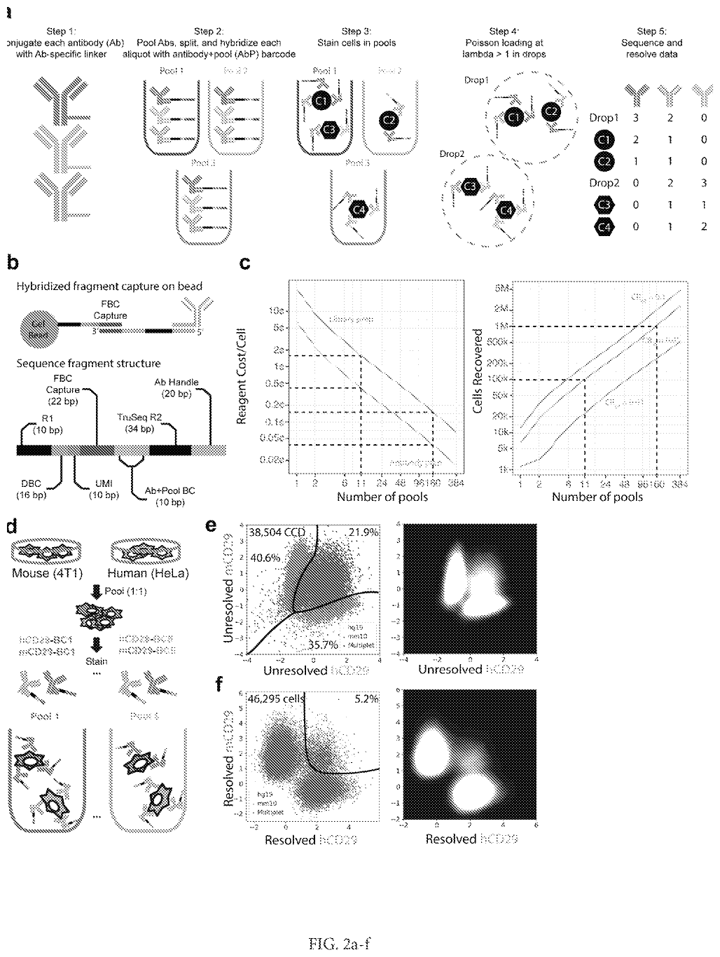

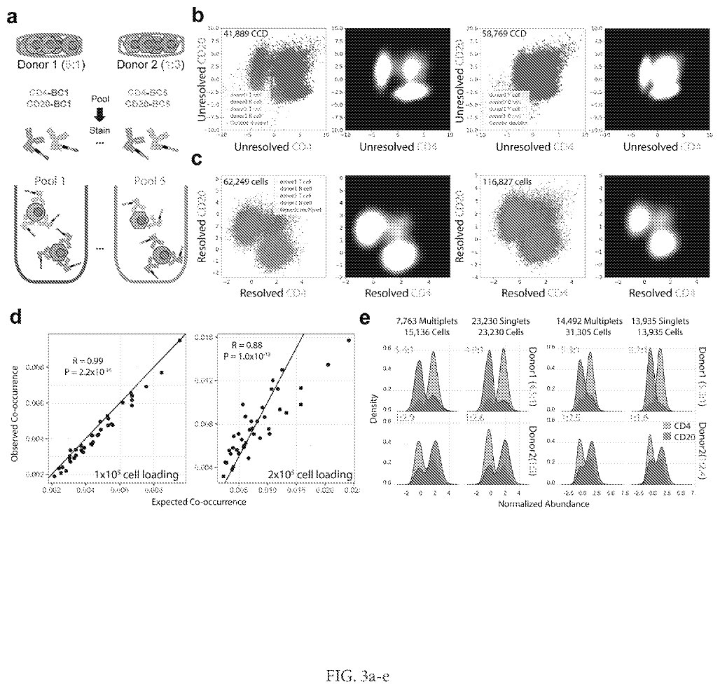

The Pool-Oligonucleotide, also referred to as “Pool Oligo,” “Splint Oligo,” “Secondary Oligo,”.and “Ab-Pool Oligo” has the structure and elements listed below. Particular embodiments of the Pool Oligo are shown in FIGS. 1 and 2. Segments include:

A “Handle Complement” (H′), an oligonucleotide sequence complementary to the Handle sequence. In one approach, the Handle Complement is at the 5′ end of the Pool Oligo. In one approach, the Handle Complement is at the 3′ end of the Pool Oligo. The Handle sequence (or its complement) sometimes has a length of about 20 bp, and usually has a length of 10 to 100 bp, and often 15 to 50 bp.

Elements for connecting the pool oligonucleotide to the droplet olionucleotide. In a hybridization-based approach a “Capture Complement” (C′) which is an oligonucleotide sequence complementary to the capture sequence of the Droplet Oligonucleotide (discussed below). In one approach, the Capture Complement is positioned at the 3′ end of the Pool Oligo is used. The Capture Complement (or Capture sequence) sometimes has a length of about 22 bp, and usually has a length of 10 to 100 bp, and often 15 to 50 bp. In a ligation-based approach the Pool Oligo has a ligatable (e.g., phosphorylated) 5′ terminus that can be ligated to the 3′-terminus of the Droplet Oligonucleotide. Advantageously ligation is facilitated by a Bridge Oligonucleotide (discussed below).

A “Pool Barcode Complement” (PBC′) or “Pool Barcode” is a barcode sequence that identifies the individual pool in which Handle-Tagged Antibodies are combined with Pool Oligos (i.e., Ab-Pool Oligos). For example, the Handle-Tagged Antibodies may be combined with Pool Oligo associated with the Handle-Tagged Antibody.

An “Antibody Barcode Complement” (ABC′) is a sequence that (like the Handle) corresponds to (identifies) the antibody portion of the Handle-Tagged Antibodies.

The “Pool Barcode” and “Antibody Barcode” may be independent barcodes including, for example, barcodes separated by an intervening non-barcode sequence. Alternatively the “Pool Barcode” and “Antibody Barcode” may be a unitary or compound barcode (e.g., a single barcode of contiguous bases that identifies both the pool and antibody. Pool barcodes can also serve as sample barcodes to enable multiplexed SCITO-seq. The choice of separate or compound Pool and Antibody Barcodes will depend on the preferences of the operator. A compound Ab+Pool barcode of a given length (e.g., 10 bp) can encode a larger number of bar code species than separate Pool and Antibody Barcodes with the same total length (e.g., 5 bp each). A compound Ab+Pool barcode often has a length of about 10 bp, such as 5 to 25 bp. The compound Antibody+Pool barcode can be referred to as an “Ab+Pool BC” or complement thereof. However, unless otherwise clear from content, any reference to the Pool Barcode and Antibody Barcode should be understood to refer equally to the compound barcode.

The Pool Oligo may optionally include other sequence features, including an amplification primer binding site or a sequencing primer binding site (which may be the same or different) shown in FIG. 2 as R2′. See discussion below.

### 5. Droplet Oligonucleotide

The “Droplet oligonucleotide” has the structure and elements listed below. Certain features of the Droplet oligonucleotide vary based on the sequencing platform used. For example, in droplet-based approaches such as 10X Genomics Chromium, inDrop and Drop-seq (see Zhang et al., 2019, Comparative Analysis of Droplet-Based Ultra-High-Throughput Single-Cell RNA-Seq Systems, Molecular Cell 73:130-142.e5, incorporated herein by reference), multiple copies of a Droplet oligonucleotide (generally having the same, unique, sequence) are attached to a bead or similar solid substrate compatible with droplet-based analyses (shown as a circle in FIG. 1 and FIG. 2). In micro-well based systems multiple copies of a Droplet oligonucleotide (generally having the same, unique, sequence) are introduced into a microwell. See Fan et al., 2015, Expression profiling. Combinatorial labeling of single cells for gene expression cytometry Science, 347:1258367; Han et al., 2018, Mapping the mouse cell atlas by Microwell-seq, Cell, 172:1091-1107.e17. As used herein, “same, unique, sequence” means that, exclusive of the UMI, if present, the Droplet Oligonucleotides in any droplet or well are different from sequences of the Droplet Oligonucleotides in the vast majority (greater than 95%, sometimes greater than 99%) of other wells or droplets.

Specific embodiments of the Droplet Oligonucleotide are shown in FIG. 1 and FIG. 2. Droplet Oligonucleotide segments include:

A “Capture Sequence” region (C) for association with the Pool Oligonucleotide. Typically the capture sequence is at the 3′ end of the Droplet oligonucleotide. In a hybridization-based approach, the Capture Sequence may be complementary to the Capture Complement of the Pool Oligo. Alternatively, in a ligation-based approach the 3′ terminus of the Droplet Oligo is joined to a ligatable end of the Pool Oligonucleotide (e.g., the 3-prime end of the Droplet Oligonucleotide may be ligated to a phosphorylated 5′ end of the pool oligonucleotide.)

A “Droplet barcode” (DBC) sequence, which is typically 5′ to the Capture Sequence. The DBC is configured so that there is one DBC sequence per compartment (discussed below). In bead-based systems each bead is associated with a unique DBC (represented as many copies in or on the bead). In well-based systems each well contains multiple copies of a well-specific BC. The term “Droplet barcode” does not require that the compartment be a droplet.

The Droplet oligonucleotide may contain additional barcodes, such as a unique molecular identifier or UMI.

The Droplet oligonucleotide typically include other features, such as amplification primer binding sites or sequencing primer binding sites (which may be the same or different) shown in FIG. 1 and FIG. 2 as R1 and in FIG. 6A as p %, for example. See discussion below.

### 6. Cells and CSP Panels

The SCITO assay is used to characterize the distribution of multiple CSPs in a cell population, and therefore uses a panel of multiple Handle-Tagged Antibodies. In various embodiments the number of different CSPs for which there are Handle-Tagged Antibodies in an assay is at least 3, at least 5, at least 10, at least 12, at least 15, at least 10, or at least 25 such as, for example, from 3 to 100, from 5 to 50, from 10 to 50, from 15 to 50, or from 25 to 50.

Exemplary panels for human immune cells include:
i) CD8, CD56, CD19, CD20, CD11c, CD14, CD33
ii) CD8, CD56, CD19, CD20, CD11c, CD14, CD33, CD66b, CD34, CD41, CD61, CD235a, CD146
iii) CD45, CD33, CD3, CD19, CD117, CD11b, CD4, CD8, CD11c, CD14, CD127, FceR1, CD123, gdTCR, CD45RA, TIM3, PD-L1, CD27, CD45RO, CCR7, CD25, TCR_Va24_Ja18, CD38, HLA_DR, PD-1, CD56, CD235, CD61

As noted above, any type(s) of cells may be used in the assay. Generally a sample contains is a heterogeneous mixture of multiple cells types (e.g., peripheral blood cells) or a heterogeneous mixture of similar cells exposed to different conditions, having different developmental histories, or the like. Cells used in the assay may be prepared by known means (e.g., washing, optional fixation).

### 7. Workflow—Pooling and Splitting the Panel

A panel of Handle-Tagged Antibodies representing the CSPs being assayed is selected and the Handle-Tagged Antibodies are pooled into a single mixture (“panel pool”). Generally the panel pool contains equal amounts of each represented antibody. However, the relative proportions of individual Handle-tag antibodies can vary and can be selected by the practitioner based on the cell population, the affinity of different antibodies for the corresponding antigen, etc.

The number of different Handle-Tagged Antibodies, exclusive of controls, may be equal to the number of surface proteins being assayed for.

As illustrated in FIG. 2 “Step 2”, the mixture of pooled Handle-Tagged Antibodies is divided or aliquoted into a plurality of vessels, typically resulting in the same combination and quantity of Handle-tagged antibodies in each vessel. It will be appreciated that, merely for clarity, this disclosure adopts the convention that step 2, shown in FIG. 2, involves aliquoting into “vessels” and step 4, shown in FIG. 2, involved dividing into “compartments” (e.g., droplets). These separate terms are not intended to limit either step to particular types of containers or mechanisms of dividing.

### 8. Workflow—Distributing Pool Oligos

As illustrated in FIG. 2 “Step 2”, aliquots of the combined Handle-Tagged Antibodies are distributed to separate vessels or “pools.” Each separate pool is combined with pool-specific Pool Oligonucleotides such that each different vessel receives a set of Pool Oligonucleotides that share the same Pool Barcode. The terms “Pool Oligonucletides” and “Splint Oligonucleotides” are used interchangeably. The two components can be introduced into the compartments simultaneously or in either order—that is the Handle-Tagged Antibodies can be added to vessels containing Pool Oligos, Pool Oligos can be combined with vessels containing Handle Tagged Antibodies, or they can be combined simultaneously. As noted, each vessel/aliquot/pool receives a different set of Pool Oligonucleotides. As noted above, in one approach titrated antibodies are mixed and aliquoted before the addition of splint oligos.

The Handle complement sequences of the Pool Oligos and Handle sequences of the Handle-Tagged Antibodies are allowed to anneal in the vessel to form the “Staining Construct.” As a result, each pool or compartment contains Pool Oligos that have a common Pool Barcode (which identifies the pool), and contains Antibody Barcodes, Handle sequences, and Handle Complement sequences all of which identify the antibody specificity of the Handle-Tagged Antibody. In one approach, the Handle is attached at its 3′ terminus to the antibody (see, e.g., FIG. 1). In another approach the Handle is attached at its 5′ terminus to the antibody (see, e.g., FIG. 6A). It will be understood that the Handle Complement will have an antiparallel orientation to the Handle. As illustrated in FIG. 1 (bottom) the position of the Handle complement in the Splint Oligo can vary.

Table 2 and FIG. 2a illustrate that in an assay in which three (3) cell surface proteins are measured, each pool would contain a set of Staining Constructs (Handle-Tagged Antibody and Pool Oligo) that contain the same PBC sequence (or otherwise identify the same pool) and all combinations of Handle/Ab-bar code sequences.

TABLE 2

| Target cell | Antibody | Pool 1 contains | Pool 2 contains | Pool 3 contains |
|---|---|---|---|---|
| surface | specific | all sequences in | all sequences in | all sequences in |
| protein | for CSP | this column | this column | this column |
| CSP 1 | Ab 1 | PBC 1-ABC 1 | PBC 2-ABC 1 | PBC 3-ABC 1 |
| | | Handle 1 | Handle 1 | Handle 1 |
| CSP 2 | Ab 2 | PBC 1-ABC 2 | PBC 2-ABC 2 | PBC 3-ABC 2 |
| | | Handle 2 | Handle 2 | Handle 2 |
| CSP 3 | Ab 3 | PBC 1-ABC 3 | PBC 2-ABC 3 | PBC 3-ABC 3 |
| | | Handle 3 | Handle 3 | Handle 3 |

It will be recognized that when a unitary or compound Pool Barcode-Antibody Barcode (Ab+PBC) is used, each pool or compartment contains Pool Oligos containing compound Pool Barcode-Antibody Barcode in which all identify the Pool and subsets identify the Antibody.

It will be recognized that it is not required that all of the Pool Barcodes (or Pool-identifying portions of the unitary Pool Antibody Barcode) in a vessel are necessarily the same (i.e., identical sequence) so long as the pool is identified by the sequence.

### 9. Workflow—Stain Cells in Pools/Vessels and Pool Stained Cells

A plurality of cells is added to each well, whereby the cells in each well are stained with (bound by) the Staining Constructs. Thus, each cell displaying a CSP(s) is bound to one or more Staining Constructs containing an antibody-specific Handle and antibody specific barcode (PBC′) and a pool barcode (ABC′).

In one approach, cells are combined with Handle-Tagged antibodys (HTAs) prior to adding Pool Oligos. Pool Oligos may be added after HTAs have bound cells. Alternatively, cells, HTAs and Pool Oligos can be combined at the same time and self assemble to produce stained cells. These approaches may have advantages in certain microfluidic work-flows, but are likely to result in increased background. Generally, as discussed above, HTAs and Splint Oligos are allowed to associate to form a complex prior to being combined with cells.

Following staining, the stained cells may be combined into a mixture prior to distribution into compartments.

### 10. Compartmentalization Platforms

The compositions and methods of the invention can be carried out using droplet-based methods, including the InDrop, Drop-seq, 10× Genomics Chromium platforms and non-droplet based methods as discussed in § 5 above. See Zhang et al., 2019, Comparative Analysis of Droplet-Based Ultra-High-Throughput Single-Cell RNA-Seq Systems, Molecular Cell 73:130-142.e5; Mimitou et al., 2019, Multiplexed detection of proteins, transcriptomes, clonotypes and CRISPR perturbations in single cells Nature Methods 16:409-412; Fan et al., 2015, Expression profiling. Combinatorial labeling of single cells for gene expression cytometry Science, 347:1258367; and Han et al., 2018, Mapping the mouse cell atlas by Microwell-seq, Cell, 172:1091-1107.e17, each of which is incorporated herein by reference. In general, reagents and methods described in the literature or materials from manufacturers can be adapted to the present invention.

### 11. Workflow—Loading of Compartments

According to the present invention, the stained cells are pooled and distributed into wells or droplets. Loading cells can be carried out using art known means including using commercially available devices used for droplet-based single cell sequencing. See, e.g., Section 10.

Conventional cell analysis methods generally require that individual cells are contained in separate compartments, typically according to a Poisson distribution. For example, the 10× literature recommends steps to maximize the number of droplets that have a single cell (single cell encapsulation), and minimize the number of droplets that are empty or contain two or more than two cells. See Zheng et al., 2017, Massively parallel digital transcriptional profiling of single cells Nature Communications 8, Article number: 14049 and kb.10xgenomics.com/hc/en-us/articles/218166923-How-often-do-multiple-Gel-Beads-end-up-in-a-partition. For the 10X Genomics platform, Poisson loading at the recommended concentrations of 2×10³-2×10⁴ cells result in collision rates of 1-10%. However, greater than 97%-82% of droplets do not contain a cell, leading to wasted reagents. In contrast, according to the present methods, antibody binding to CSPs from two cells, or two or more cells, in the same droplet (multiplets) can be distinguished and resolved based on the information provided by barcodes. In the present methods cells may be loaded at high concentrations where the majority of droplets will contain at least one cell. tunable to a targeted collision rate. For example, for a commercially available microfluidic platform where ^{˜}10⁵ droplets are formed, a loading concentration of 1.82×10⁵ cells results in 84% of droplets containing at least one cell but only 4.4% of droplets containing greater than four cells. To yield 10⁵ resolved cells at a collision rate of 5% for this loading concentration, 11 antibody pools would be needed. At 160 pools and 5% collision rate, 1×10⁶ cells can be profiled in one microfluidic reaction with an average of 18.9 cells captured per droplet. In some embodiments at least 25% of compartments occupied by at least one cell (i.e., not empty) contain two cells, sometimes at least 30%, at least 40%, at least 50%, or at least 60%. In some embodiments at least 25% of occupied compartments contain more than one cell (i.e., two or more cells), sometimes at least 30%, at least 40%, at least 50%, or at least 60%. It will be apparent that, in relation to the number of cells in a compartment or droplet, there is an upper limit beyond which benefits diminish. This in some embodiments the multiplicities of encapsulation (MOE) or number of cells per occupied compartment range from 1 to 10 cells per droplet, e.g., up to 10, up to 9, up to 8, up to 7, up to 6, up to 5, or up to 4

### 12. Production of Sequence Fragment, Sequence Determination and Sequencing Platforms

As illustrated in FIG. 1 and FIG. 2a, the Handle-Tagged Antibody, Droplet Oligonucleotide and Pool Oligo assemble to form a three-component construct in which the Capture Sequence C anneals to the Capture Complement C′, and the Handle sequence H anneals to the Handle Complement H′ as illustrated in FIG. 1 and FIG. 2a. According to one embodiment of the invention, at least a portion of the three-component construct is extended or made double stranded using art-know methods such that the DBC, PBC, and ABC, or the complements thereof are all contained in one polynucleotide, which may be single-stranded or double-stranded polynucleotide (generally DNA). STRUCTURE I, below, illustrates an organization of single, optionally double stranded, polynucleotide (the “Sequence Fragment Structure” as shown in FIG. 2b) that contains all of the segments of the three-component construct shown in FIGS. 1 and 2a. Structure 1 is provided for illustration and not for limitation.

| | | | | | | | |
|---|---|---|---|---|---|---|---|
| Primer | DBC | UMI | Capture | PBC | ABC | Primer | Handle |

### Structure I

In another approachAs illustrated in FIG. 6a, the Handle-Tagged Antibody, Droplet Oligonucleotide and Pool Oligo assemble to form a three-component construct in which the Droplet Oligonucleotide (C) is ligated to the Splint Oligo, and the Splint Oligo is hybridized to the antibody Handle.

In addition to the DBC, PBC, and ABC (sometimes referred to as “the three barcodes”) the Sequence fragment structure will include elements that allow sequencing of the three barcodes. The three barcodes can be sequenced in a single read, as two paired-end reads (also called mate pair reads), or any other fashion that identifies the combinations of the three barcodes associated on any Sequence Fragment Structure. For example, referring to FIG. 1 (lower panel), sequencing-by-synthesis from a primer hybridized to one of the two primer binding sites shown could be used to determine the three barcodes. Alternatively one primer hybridized to the Primer 1 primer binding site could be used to produce one read that identifies the DBC, a second primer hybridized to the Primer 2 primer binding site could be used to produce a second read identifying the PBC and ABC (e.g., the compound Ab+Pool BC) and the two reads associated.

It will be within the ability of a person of skill in the art to generate a sequenceable Sequence Fragment Structure using enzymes such as reverse transcriptase, DNA polymers, DNA ligase and art-known strategies such as primer extension, and to prepare a sequencing library. Sequencing may be carried out using any suitable massively parallel sequencing platform, including, for example, Illumina's cluster based sequencing by synthesis platforms and MGI's DNBSeq platforms.

### 13. Analysis and Deconvolution

Using the present invention, data from each individual cell includes three identifiers (barcodes): Handle-Tagged Antibody, Pool Oligonucleotide, Droplet Oligonucleotide, and optionally UMI data. As discussed below, using this approach the surface protein expression profiles of multiple encapsulated cells (multiplets) within a droplet can be resolved by the combinatorial index of Antibody Barcode, Pool Barcode (e.g., Ab+PBC) and Droplet Barcode.

### 14. SCITO Theory, Design and Demonstrations

As cell loading is governed by a Poisson distribution, the major limitation of standard droplet-based single cell sequencing (dsc-seq) workflows is ensuring encapsulation of single cells to reduce the number of collisions. This results in suboptimal cell recovery, reagent usage, and inflated library construction costs. For the 10X Genomics single-cell sequencing platform, Poisson loading at the recommended concentrations of 2×10³-2×10⁴ cells result in cell recovery rates (CRR) of 50-60%^{16,22} and collision rates of 1-10%. However, at these concentrations, 97%-82% of droplets do not contain a cell, leading to wasted reagents. One approach to decrease the library preparation cost and increase the sample and cell throughput of dsc-seq is to “barcode” samples using either natural genetic variants^{10,23,24} or synthetic DNA molecules^{11,12,25} prior to pooled loading at 5×10⁴-8×10⁴ cells, reducing the proportion of droplets without a cell to ^{˜}65%-45%. Because simultaneous encapsulation of cells within a droplet can be detected by the co-occurrence of different sample barcodes (e.g., genetic variant or synthetic DNA tags) with the same droplet barcode (DBC), sample multiplexing increases the number of singlets recovered per microfluidic reaction while maintaining a low effective collision rate tunable by the number of sample barcodes. However, since collision events can only be detected but not resolved into usable single-cell data, the maximum loading concentration that minimizes total cost is ultimately limited by the overhead cost incurred for sequencing collided droplets.

Single-cell combinatorial indexing (SCI) is an alternative, scalable approach to control the collision rate of single-cell sequencing by labeling subsequent rounds of physical compartmentalization with DNA barcodes. While standard SCI approaches require more than two rounds of combinatorial indexing to sequence 10⁵-10⁶ cells¹⁷⁻²⁰, recent advances utilizing droplet-based microfluidics for combinatorial indexing have enabled simplified two-round workflows to achieve the same throughput^{21,22}. For applications where only a set of targeted markers are needed such as high-throughput screens and clinical biomarker profiling, current SCI workflows profiling the entire epigenome or transcriptome per cell is not optimized for sensitivity and would likely result in prohibitively high sequencing costs.

An element of SCITO-seq arises from the recognition that Poisson loading naturally limits the number of cells within a droplet even at very high loading concentrations. Thus, indexing cells using a small number of antibody pools will ensure that the combinatorial index (Ab+PBC and DBC) will identify a cell at low collision rates even at high loading concentrations. Theoretically, given P pools, C cells loaded, D droplets formed, the collision rate is given as

$$[ Collision ] = 1 - {{e}^{- \frac{C}{D}} [ 1 + \frac{C}{PD} ]}^{P}$$

while rate of empty droplets is given by

$$[ Empty ] = {e}^{- \frac{C}{D}}$$

(see § 23, Methods). Our derivation of the collision rate differs from previously reported estimates derived from the classical birthday problem²², which did not account for higher order collision events of more than two cells with the same barcode. These closed form derivations of the collision and empty droplet rates are nearly identical to those obtained based on simulations. For example, when 6×10⁵ droplets are formed, a loading concentration of 1.82×10⁵ cells (target recovery of 10⁵ cells) results in 84% of droplets containing at least one cell but only 4.4% of droplets containing greater than four cells. To yield 10⁵ resolved cells at a collision rate of 5% for this loading concentration, only 10 antibody pools would be needed to achieve a total cost of 3.1¢/cell. Note that as the library preparation cost quickly diminishes for SCITO-seq with increasing number of pools, the total cost per cell is dominated by antibody costs. Therefore, while 384 pools achieves the maximal 12-fold reduction in cost compared to standard single-cell proteomic sequencing (2.2 vs 26 cents), 10 antibody pools can already achieve a 8-fold reduction in cost (3.1 vs 26 cents) while minimizing experimental complexity (FIG. 2c).

To demonstrate the feasibility and scalability of SCITO-seq, we performed a mixed species experiment by pooling human (HeLa) and mouse (4T1) cells, splitting into five aliquots, and staining each pool with anti-human CD29 (hCD29) and anti-mouse CD29 (mCD29) antibodies labeled with pool-specific barcodes (FIG. 2d). After washing unbound antibodies and mixing the five stained pools at equal proportions, 10⁵ cells were loaded for ADT library construction using the 10X Genomics 3′ V3 chemistry and the resulting library sequenced to recover 38,504 post-filtered cell-containing droplets (CCDs) at a depth of 2,909 reads/CCD. For comparison purposes, we also obtained a library derived from the RNA and sequenced it to 25,844 reads/CCD. Merging ADTs for each antibody across pools to mimic standard single-cell proteomic profiling⁴, we detected 40.6% and 35.7% of CCDs with only mouse or human CD29 ADTs and 21.9% with CD29 ADTs from both species which we labeled as cross-species multiplets (FIG. 2e, see § 23, Methods). These estimates were consistent with results from analyzing the transcriptomic data: 42.7% CCDs had mouse transcripts, 33.9% had human transcripts, and 23.3% had transcripts from both species. By utilizing the DBC and Ab+PBCs combinatorial indices, we resolved both between- and within-species multiplets, reducing the collision rate from an estimated 51% to 8.8% (expected 6.3%) (FIG. 2f) without significant pool to pool variation. The ability to resolve cross and within-species multiplets results in a total of 46,295 cells profiled at an estimated collision rate of 11.4%, a 3.7-fold increase over standard workflows (12,500 cells at 11.6% collision rate) (FIG. 2f). Further, we observed that a two-pool SCITO-seq experiment produced similar results to an alternative design using direct conjugation of four different Ab+PBC barcodes suggesting that both within and between pool splint oligo contamination rates are low and sensitivity is retained across direct and hybridized conjugates.

### 15. Scito-Seq is Scalable to >100K Cells and Captures Compositional Shifts

We next sought to further assess the scalability of SCITO-seq and its applicability to resolve quantitative differences in cellular composition based on surface protein expression. We isolated and mixed primary CD4+ T and CD20+ B cells from two donors at a ratio of 5:1 (T:B) for donor 1 and 1:3 (T:B) donor 2. The mixed cells were aliquoted into five pools and each stained with pool-barcoded anti-CD4 and anti-CD20 antibodies (FIG. 2g). Stained pools were mixed at equal ratios, loaded at 2×10⁵ cells per channel on the 10X Chromium system, processed with 3′V3 chemistry, and the resulting ADT and RNA libraries sequenced to recover 58,769 post-processing CCDs.

Merging the ADT data across the five pools, anti-CD4 and anti-CD20 antibodies stained the expected cell types defined by the transcriptome. Based on the ADTs, we estimated 40% of CCDs to be between cell-type multiplets, which is consistent with estimates from the transcriptomic analysis (49.6%, FIG. 2h). We further used genetic demultiplexing (www.github.com/statgen/popscle) to leverage genetic variants captured in the transcriptomic data to estimate 30% within cell-type multiplets for a total multiplet rate of 70%. After resolving both between and within cell-type multiplets using the combinatorial index of Ab+PBC and DBC with minimal pool to pool variation, we reduced the collision rate from an estimated 70% to 25%. A total of 116,827 resolved cells were profiled, effectively increasing the throughput by 4.0-fold over standard workflows at the same collision rate. Note that both the multiplet rates (R=0.97, P<0.01) and the co-occurrence rates of SCITO-seq antibodies from different pools (R=0.93, P<0.01) were highly correlated between the expected and observed values. These results suggest that the encapsulation of multiple cells within a CCD is not biased for specific pools or cell types.

We next assessed if SCITO-seq can capture unequal distributions of B and T cells from the two donors, especially from CCDs that encapsulated multiple cells. For this analysis, we focused only on 45,240 CCDs (donor 1: 25,630, donor 2: 19,610) predicted to contain cells from only one donor based on genetic demultiplexing. Within CCDs with only one antibody pool barcode detected, analysis of the proportions of T and B cells (T:B200K:5.0:1 for donor 1 and 1:2.8 for donor 2) mirrored the expected proportions for each of the two donors and was consistent with estimates obtained from the transcriptomic data. Encouragingly, approximately the same proportions were estimated in CCDs with multiple pool barcodes (multiplets) (T:B200K 4.0:1 for donor 1 and 1:2.9 for donor 2).

Because pool-specific effects appear to be minimal in SCITO-seq, the pool-specific antibody barcodes could be used to directly label samples, obviating the need for orthogonal sample barcoding. To demonstrate this application, we performed another experiment where we stained one donor per pool and each pool contained different barcoded antibodies (e.g., pool 1 contains CD4-BC1 while pool 2 contains CD4-BC2, etc.). For loading concentrations of 2×10⁴ and 5×10⁴ cells, we obtained 17,730 and 34,549 post-processing CCD, sequenced to a per CCD depth of 964 and 1,540 reads for the ADT and 20,951 and 14,332 reads for the RNA. We observed the expected proportion of T and B cells per donor based on the distribution of the expression of CD4 and CD20 respectively. After resolution, we recovered 18,680 and 41,059 cells at collision rates of 7.4% and 18.6% respectively. Estimates of co-occurrence frequencies of different pool and antibody barcodes were highly correlated (r=0.99, p-value<0.001) with observed values.

### 16. Scito-Seq Quantifies Donor Specific Composition in PBMCS Consistent with Cytometry

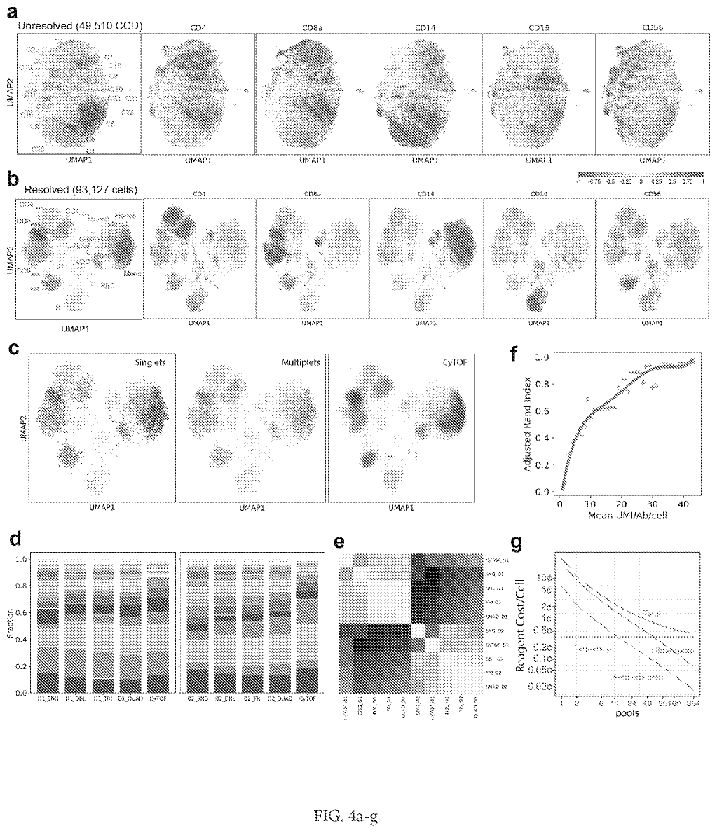

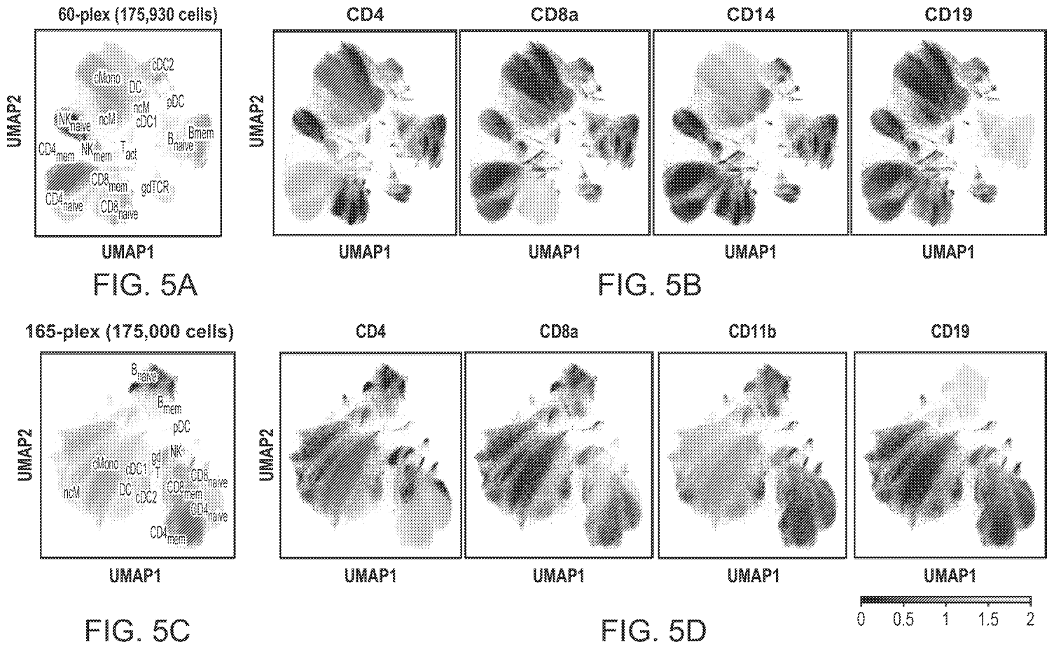

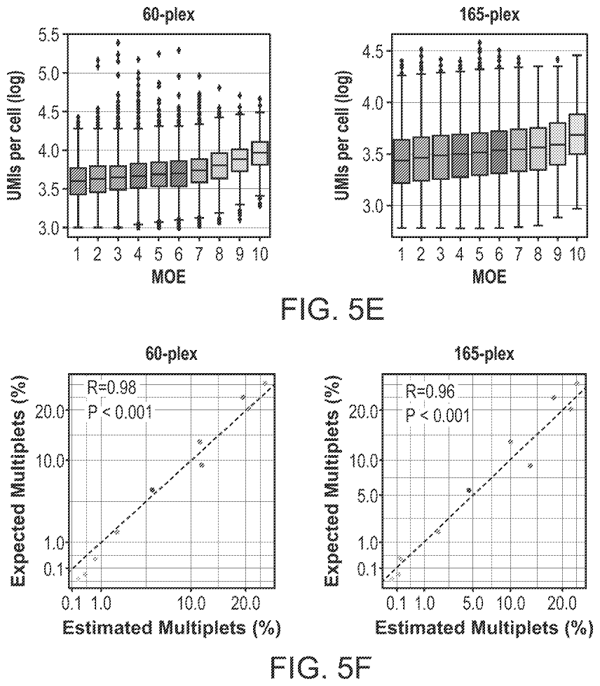

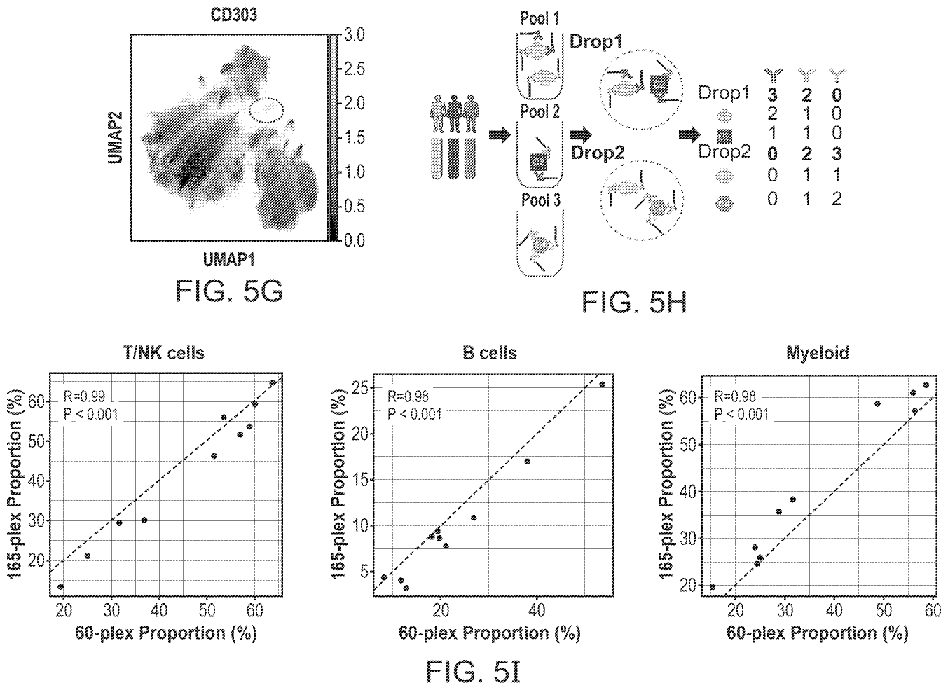

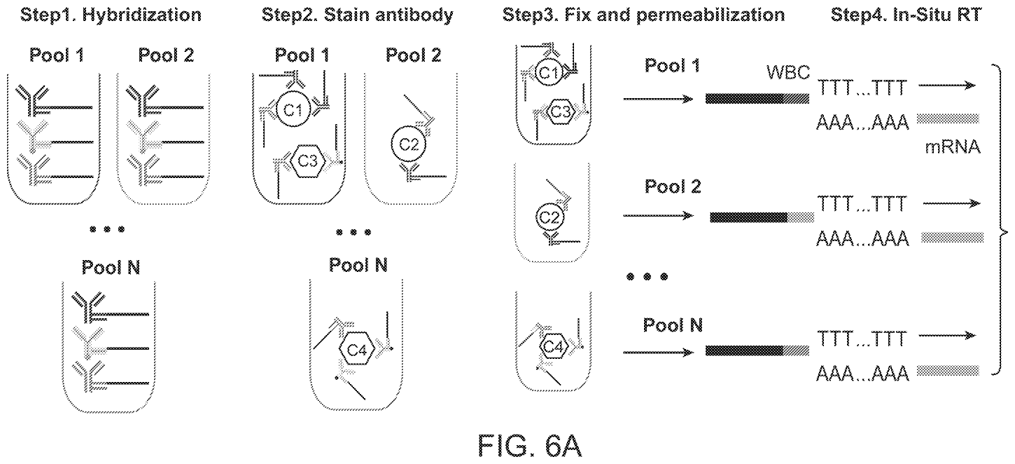

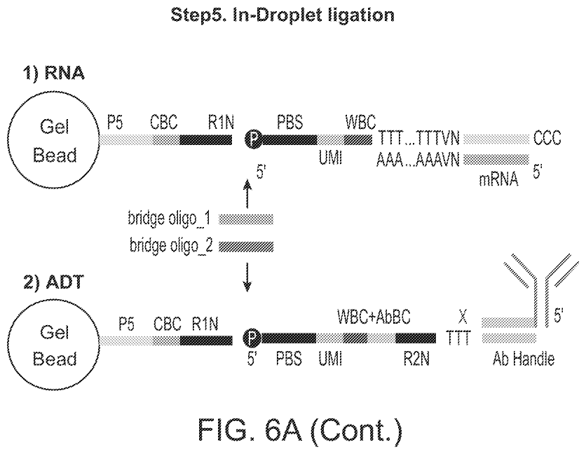

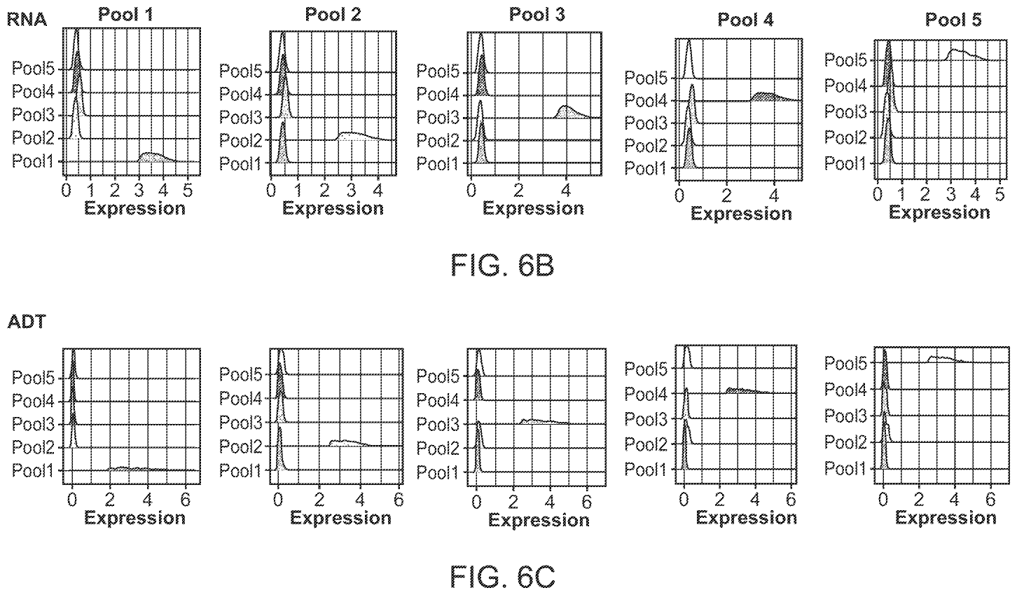

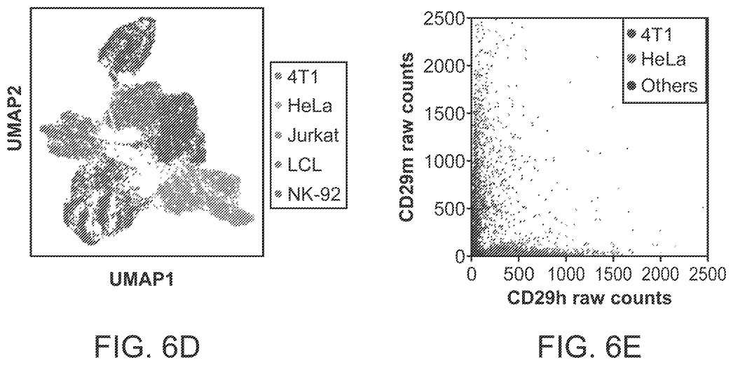

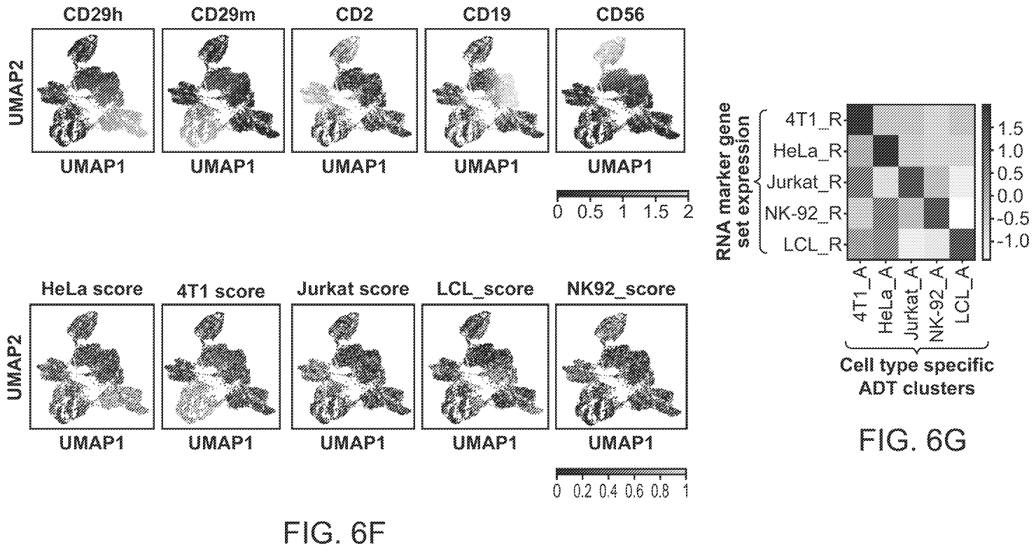

To demonstrate SCITO-seq's applicability for high-dimensional and high-throughput cellular phenotyping, we profiled peripheral blood mononuclear cells (PBMCs) from two healthy donors using a panel of 28 monoclonal antibodies across 10 pools. After staining, pooling, and processing 2×10⁵ cells in a single 10X channel using 3′V3 chemistry, we sequenced the resulting ADT and RNA libraries and obtained 49,510 post-filtering CCDs (FIG. 4a). Each of the 10 SCITO-seq pool barcodes was detected in a subset of CCDs at levels significantly different from other pool barcodes suggesting a high signal-to-noise ratio to resolve multiplets. In total, we resolved 93,127 cells at a collision rate of 8.5%, increasing the throughput by 10-fold over standard workflows at the same collision rate consistent with the simulations.

We separately analyzed the merged ADT and RNA data by normalizing the counts, performing dimensionality reduction, and constructing a k-nearest neighbor graph (see § 23, Methods). Leiden clustering based on either merged ADT or RNA counts (FIG. 4a) resulted in clusters that were poorly differentiated in Uniform Manifold Approximation and Projection (UMAP) space due to the high multiplet rates (69%) at these loading concentrations. Encouragingly, Leiden clustering using resolved ADT counts resulted in 17 distinct clusters in UMAP space which could each be annotated based on the expression of lineage specific ADT markers (FIG. 4b). We detected eight clusters of the myeloid lineage, naïve and memory CD4+ and CD8+ T cells, natural killer (NK) cells, B cells and gamma delta T cells (gdT). Notably, naive (CD45RA+) and memory (CD45RO+) CD4+ and CD8+ T cells emerge as separate clusters which can often be difficult to distinguish based on the RNA data due to low transcript abundances of lineage markers (e.g. CD4) and inability to infer isoforms (e.g. CD45RO)¹⁶. Indeed, analyzing the transcriptomes of CCDs likely containing only a single cell (see § 23, Methods) shows limited separation of naive and memory CD4+ CD8+ T cells when compared to overlaid antibody expression.

We further assessed the accuracy of SCITO-seq for quantitative immune phenotyping by comparing the compositional estimates obtained from CCDs with a single detected pool barcode (singlets) versus those with multiple detected pool barcodes (multiplets). We focused the analysis only on CCDs with cells from one donor as estimated using genetic multiplexing. UMAP projections for resolved cells originating from singlets vs multiplets were qualitatively similar (FIG. 4c), suggesting that higher rates of encapsulation do not create technical artifacts in the data. We quantitatively confirmed that the frequency estimates of the 16 immune populations detected from singlets and multiplets (doublet, triplet, quadruplets) were more similar from the same donor (average cosine similarity (CS): 0.98 [donor 1], 0.97 [donor 2]; FIGS. 4d and 4e) than between different donors (average CS: 0.83). To orthogonally evaluate the data produced by SCITO-seq, we performed mass cytometry (CyTOF) using the same antibodies conjugated to metal isotopes. Joint clustering of the CyTOF and SCITO-seq data produced qualitatively similar UMAP projections (FIG. 4c) and the frequency estimates of jointly annotated cell types were highly similar between assays for the same donor (average CS: 0.95 [donor 1], 0.93 [donor 2]) (FIG. 4e).

One advantage of SCITO-seq as a tool for high-dimensional and high-resolution phenotyping is the high information content obtained by profiling protein abundance. This is demonstrated by downsampling of the 2×10⁵ dataset where only ^{˜}25 UMIs/cell corresponding to ^{˜}60 reads/cell (assuming 45% library saturation) were needed to achieve an Adjusted Rand Index (ARI) of >0.8 for assigning cells to the same clusters in the full dataset (FIG. 4f). A similar trend was observed for the data from 1×10⁵ cell loading data. As library preparation cost quickly diminishes with increasing number of pools, the total cost per cell is dominated by sequencing and by sequencing a limited number of targets, SCITO-seq remains cost effective even when large numbers of pools are used (FIG. 4g). The cost-effectiveness, simple design and potential for incorporating additional modalities and orthogonal experimental information position SCITO-seq well as a new method for scalable high-dimensional phenotyping, especially for applications such as high-throughput screening and clinical biomarker profiling where targeted profiling of a limited set of markers is needed.

### 17. Scaling Scito-Seq to Large Custom and Commercial Antibody Panels

To further demonstrate the flexibility and scalability of SCITO-seq beyond the number of markers detectable by competing flow and mass cytometry methods^{9,26}, we evaluated the performance of SCITO-seq using a 60-plex custom panel and a commercial Totalseq-C(TSC) 165-plex antibody panel. To achieve compatibility with the commercial TSC panel where anti-body oligos are conjugated on the 5′ end versus the 3′ end for SCITO-seq, we designed a set of splint oligos to hybridize to each of the 165 15 bp antibody barcodes in the panel.

For both experiments, we further leveraged the pool barcodes encoded in each set of splint oligos as a sample label to enable multiplexing. We stained the same 10 donors in 10 distinct pools using either panel and loaded 4×10⁵ cells to tune our targeted recovery to 2×105 cells per experiment. In the 60-plex experiment, we recovered 69,733 CCDs and resolved 219,063 cells (FIG. 5a, 5b) with a collision rate of 18.7%. In the 165-plex experiment, we recovered 66,774 CCDs and resolved 203,838 cells (FIGS. 5c and 5d) at a collision rate of 14.1%. Note that even at a loading concentration of 4×10⁵ cells, 20-fold higher than recommended, we did not observe a plateau for the number of UMIs recovered versus the number of cells per CCD suggesting that reagents are not yet a limiting factor (FIG. 5e). In addition, we report high correlation (60-plex; R=0.99, P-value<0.001, TSC; R=0.92, P-value<0.001) between simulated and observed multiplet rates (FIG. 5f).

After removal of collided barcodes based on the number of expressed markers (see § 23, Methods), we obtained 175,930 and 175,000 cells in the 60-plex and 165-plex experiments respectively. After normalization, dimension reduction, and k-nearest neighbor graph construction, the cells were clustered into 26 and 19 clusters respectively and visualized in UMAP space (FIG. 5a, 5c). The expected lymphoid and myeloid cell types were annotated with lineage markers (FIG. 5b, 5d). Compared to the 28-plex dataset, higher dimensional phenotyping enabled the identification of low frequency cell types such as two populations of conventional dendritic cells (cDC1s and cDC2s) distinguished by the expression of CD141, CD370, CD1c and plasmacytoid dendritic cells (pDCs) by the expression of CD123, CD303 and CD304²⁷ (FIG. 5a, 5c, 5g).

The increase in throughput of SCITO-seq can be particularly useful for large-scale profiling of multiple samples. This is further facilitated by the pool barcodes in the splint oligo design which can be used to directly label samples obviating the need for orthogonal sample barcoding (FIG. 5h). We performed a pairwise analysis across all antibodies for both experiments and observed no significant correlation across batches. This result, in addition to our previous observation of minimal pool-specific effects suggests the feasibility of using pool-specific antibody barcodes for sample labeling (FIG. 5h). Verifying the performance of multiplexed SCITO-seq, we observed high correlation in the compositional estimates across various (T, NK, B, and Myeloid) immune cell populations (R=0.98-0.99, P-value<0.001) between experiments for the same ten donors (FIG. 5i).

### 18. Combinatorially Indexed Transcriptomic and Proteomic Profiling

We sought to enable combinatorially indexed multimodal profiling of the transcriptome and surface proteins by combining SCITO-seq with the recently published scifi-RNA-seq²². Scifi-RNA-seq generates combinatorial indices by adding pool-specific barcodes on transcripts through in-situ reverse transcription and ligates the DBC from the 10X single-cell ATAC-seq (scATAC-seq) gelbeads. See Datlinger et al., 2019, Ultra-high throughput single-cell RNA sequencing by combinatorial fluidic indexing, bioRxiv, incorporated herein by reference. To first enable compatibility of SCITO-seq with the scATAC-seq chemistry, we modified the bead hybridization sequence of the splint oligo to be complementary to the ATAC-seq gelbead sequence. After droplet emulsion breakage and subsequent harvest with silane DNA-binding beads, DNA was eluted and amplified to add sequencing adaptors. We applied the modified SCITO-seq workflow to profile PBMCs from one donor in five pools with 12 broad phenotyping surface markers using the 10X scATAC-seq chemistry. As a proof of principle, we loaded 5×10⁴ cells to recover 21,460 cells and identified the expected clusters of T, B, myeloid, and NK cells expressing the canonical surface proteins demonstrating the compatibility of SCITO-seq with scATAC-seq chemistry.

Scifi-RNA-seq utilizes a bridge oligo to facilitate the ligation of DBCs within scATAC-seq gelbeads and requires a number of cycling conditions that is not directly compatible with SCITO-seq. To enable multimodal profiling, we next designed an orthogonal bridge oligo specific to the SCITO-seq design to assist capture and ligation of SCITO-seq ADTs to the 10X scATAC-seq gelbead capture sequence (FIG. 6a). This allows for a second round of indexing by an addition of a DBC without modifying the scifi-RNA-seq protocol while minimizing the competition between bridge oligo capture of transcript and ADT molecules. As a proof of principle, we applied this modified SCTIO-seq protocol to profile a mixture of four human cell lines (LCL, NK-92, HeLa, Jurkat) and one mouse cell line (4T1) with six surface antibodies in five pools prior to performing the scifi-RNA-seq workflow (FIG. 6a). We loaded 3×10⁴ cells and resolved 10,439 cells based on ADT counts. Further analysis of the distribution of cells with respect to RNA and ADT pool barcodes revealed minimal mixing of barcodes from different pools and high signal to noise ratio in resolving cells (FIGS. 6b and 6c).

After pre-processing, we obtained an average of 310 UMIs per cell for the RNA library (average 146 genes/cell) and an average of 550 UMIs per cell for the ADT library. After normalization of the ADT counts, dimensionality reduction, and k-nearest neighbor graph construction, we identified 5 clusters using Leiden clustering visualized in UMAP space (FIG. 6d). To demonstrate specificity of transcripts and antibody barcodes, we plotted the abundances of human vs mouse CD29 antibodies across all cells and observed a near equal distribution of cells expressing human vs mouse CD29 (Gini index of 0.12) (FIG. 6e). Furthermore, by aggregating sets of transcript markers specific to each cell line (see § 23, Methods), we show that expression of sets of cell type specific transcripts overlapped with the corresponding populations identified using surface protein markers (FIG. 6f). While HeLa and 4T1 specific transcripts were prominently expressed in HeLa and 4T1 ADT clusters, NK-92 specific transcripts were notably less prominently expressed in the NK-92 ADT cluster. This is likely due to the lower mRNA capture efficiency (168 UMIs per cell) for the particular cell line. To further assess congruence between the transcriptomic and ADT data, we overlaid the transcriptomic UMAP with ADT clusters to demonstrate enrichment amongst the same populations. In addition, overlap analysis (i.e. computed z-scores of sets of transcriptomic markers overlaid on ADT UMAP space) quantitatively confirmed that marker transcripts are also enriched in respective ADT clusters including NK-92 (FIG. 6g). These results demonstrate a provisional implementation of SCITO-seq that is compatible with scifi-RNA-seq and has the potential for ultra high-throughput multimodal profiling of RNA and proteins from the same cells using combinatorial indexing.

### 19. Comodality

To generate compatible secondary oligos with scifi-RNA-seq, we conjugated unique 20 bp 5′ amine modified oligos to each of our six antibodies, varying from our previous 3′ amine conjugation to present a favorable orientation of the secondary oligonucleotide (Splint Oligo) for capture in a similar fashion to transcripts in the scifi-RNA-seq workflow. In addition, we spiked-in an additional orthogonal bridge oligo for the in-emulsion ligation to reduce competition of transcripts and ADT molecules for the bridge oligo. We stained 5 pools of a mixture of 5 cell lines for 30 min prior to washing and executing the scifi-RNA-seq protocol. After the scifi-RNA-seq workflow, we loaded 3×104 into the 10× chromium controller using the lox ATAC-seq kit. After emulsion breakage as in the 10× user guide, we saved 4 μl of the 24 μl silane bead elution for ADT library construction. The ADT sample index PCR reaction was set up with 4 μl of sample, 5 μl of P5 primer (10 μM), 5 μl of i7 index primer (10 μM), 50 μl of KAPA HiFi mastermix, and 36 μl of RNAse-free water. Cycling conditions were as follows: 98° C. for 45 s, followed by 12 cycles of 98° C. for 20 s, 54° C. for 30 s, 72° C. for 20 s, and ending with a final extension of 72° C. for 1 min. We cleaned up and selected the fragments using AMPure XP beads at a ratio of 1.2X, prior to a final elution in 20 μl. To construct the gene expression library, we used a plexWell 96 Library Preparation kit (Seqwell ref PW096-1) to tagment 10 ng of DNA per reaction. This pre-loaded Tn5 was used to ease the number of tagmentations in the scifi-RNA-seq workflow and increase the reproducibility with a commerical product over custom-loaded Tn5s. The final gene expression library sample index PCR was performed as-is in the scifi-RNA-seq workflow. The resulting libraries were sequenced on a Novaseq 6000 Si v1.0 flow cell with the following read configuration: 21:8:16:78 (Read1:i7:i5:Read2).

To process the transcriptomic data, the generated fastqs (R1:21 bp, R2:16 bp, R3:78 bp) were stitched to make a final R1 file containing a droplet barcode (16 bp)+well barcode (11 bp)+UMI (8 bp) per read. We used kallisto version 0.46.1 and specified the cell barcode as 27 bp (16+11; droplet and well barcode bp lengths) and ran bustools to produce count matrices (www.kallistobus.tools/getting_started). To process the ADT fastqs (same read configuration as RNA) were stitched to produce a final R1 file (35 bp), R3 data was trimmed to 10 bp (encoding antibody barcode) for barcode alignment. These reads were then processed using a modified dropseq pipeline (v2.4.0; aligner swapped to bowtie (v2.4.2)) (www.github.com/broadinstitute/Drop-seq/releases). Counts were then normalized as done in the PBMC experiment above for both ADT and RNA. RNA genes were determined based on manual curation after running the Wilcoxon's test for determining highly variable marker genes. For overlap analysis in FIG. 6g, gene scores (using scanpy's function) for each cell lines are calculated and standardized (mean:0, variance:1, z-score to represent the classification accuracy) to be used as an input for the heatmap generation (Seaborn package's (v0.11.1) heatmap function).

### 20. Scito-Seq with the 10X ATAC-Seq Kit

We initially designed a secondary oligo compatible with the 10×ATAC-seq kit by changing the hybridizing end of the splint oligo to the reverse complement of the Read 1 Nextera sequence) from the feature barcode capture sequence (10×3′v3). We modified the microfluidic cell and enzyme mixture to the following mastermix; 4 μl of 10 mM dNTP, 16 μl of RT buffer (5×), 4 μl of Maxima H minus, and cells and RNAse free water up to 80 μl. After running the solution through a 10× chip E reaction as in the 10× user guide, the GEMs were thermocycled at 53° C. for 45 min and 85° C. for 5 min. The emulsion was broken as in the 10× user guide and ADT fragments were eluted in 40 μl. We performed an index PCR with the following conditions: 40 μl of sample, 50 μl of 2×KAPA HiFi HotStart ReadyMix, 1 μl each of P5 primer (100 uM) and universal read 2 Nextera primer, and 8 μl of RNAse-free water. The sample was cycled as follows: initial denaturation at 98° C. for 45 s, cycled 12× at 98° C. for 20 s, 54° C. for 30 s, and 72° C. for 20 s, followed by a final extension at 72° C. for 1 min.

### 21. SCITO-Seq with Commercial Antibody Panel

To scale SCITO-seq to a commerical platform, we modified our secondary oligo (Splint Oligo) to be compatible with Biolegend's TS-C platform (normally used for the 10×5′ kits) for the 10×3′V3 kit. To do this, we changed the antibody hybridization region in our original 3′v3 design to the reverse complement of antibody specific TS-C barcode (15 bp) sequences. After emulsion breakage, we followed the index PCR protocol as per manufacturer's recommendations (10× Genomics, CG000185 Rev D, page 52).

### 22. Variations and Embodiments

In additional embodiments, the Handle oligonucleotide is attached to the antibody via a noncovalent link, such as a streptavidin-biotin link, or a cleavable link, such as a disulfide bridge.

In additional embodiments, affinity reagents other than antibodies may be used to recognize CSPs. These include, for example, aptamer, affirmer, and knottins. See, e.g., U.S. Pat. No. 8,481,491; Cochran, Curr. Opin. Chem. Biol. 34:143-150, 2016; Moore et al., Drug Discovery Today: Technologies 9(1):e3-ell, 2012; Moore and Cochran, Meth. Enzymol. 503:223-51, 2012; Jayasena, et al., Clinical Chemistry 45:1628-1650, 1999; Reverdatto et al., 2015, Curr. Top. Med. Chem. 15:1082-1101. This disclosure should therefore be read as if each and every reference to “antibodies” referred equally to other “affinity reagents” not limited to aptamers, affirmers, and knottins.

In certain embodiments, some of all of the antibodies or other affinity agents to which the Handle is attached bind to cell surface proteins (e.g., peripheral membrane proteins or the extracellular portion of transmembrane proteins). In additional embodiments some or all of the antibodies or other affinity reagents used in an assay bind to any of (a) a cell-surface antigen other than a protein (e.g., cell membrane lipid); (b) intracellular proteins (e.g., cytoplasmic proteins).

The approach described herein can be use with 3′ or 5′ conjugation of the Handle to the antibody, as well as with various commercial platforms and devices. In one approach, the Handle oligonucleotide is conjugated at its 3′ end to the antibody protein as illustrated in FIG. 1 (e.g., 5′ATCG 3′-Ab). In alternative embodiments the Handle oligonucleotide is conjugated at its 5′ end to the antibody protein (e.g., 3′GCTA5′Ab). Single cell assays using oligonucleotide tagged antibodies are known in the art (see Mimitou et al., 2019, ‘Multiplexed detection of proteins, transcriptomes, clonotypes and CRISPR perturbations in single cells Nature Methods 16:409-412 (describing ECCITE-seq) incorporated by reference). A person of ordinary skill in the art, guided by the present specification, will be able to adapt the method for use with 3′ or 5′ conjugation and corresponding work flows, as well as various commercial platforms and devices. In one approach, a 5′ workflow is carried out by carried out by introducing a template switch oligo sequence (TSO) at the 3′ end of the Droplet Oligonucleotide. In one approach this can carried out by using a TSO sequence as the Capture segment (C), or a portion thereof, in the Droplet Oligonucleotide and using the reverse complement as the Capture Complement sequence in the Pool Oligonucleotide. An exemplary TSO sequence is 5′-TTTCTTATATGGG-3′. The normal 5′ workflow, e.g., as described Chromium Single Cell V(D)J Reagent Kits User Guide, Revision L to M, February 2020, Document number CG000086, incorporated by reference, can then be adapted for use in the present methods. It will be appreciated that, conjugation of the antibody at the 5′ or 3′ end of the Handle does not necessarily require conjugation at the terminal nucleotide. The antibody can be conjugated to an internal nucleotide provided the orientations of the Handle Oligo, Pool Oligo and Droplet Oligo are consistent such that the Capture Construct (comprising the three oligonucleotide components) can form, and that the antibody does not sterically interfere with formation.

It will be recognized that a pool oligonucleotide may associate with a droplet oligonucleotide by hybridization of complementary sequence or, alternatively a pool oligonucleotide may associate with a droplet oligonucleotide by ligation. In one embodiment of the ligation option the orientation of the pool oligonucleotide is reversed and there is a concomenant reversal of the orientation of the antibody handle (handle is associated with antibody at its 5′ end rather then its 3′ end. The various embodiments described in detail in this disclosure are not intended to be limiting in any fashion. The reader will recognize that rearrangements consistent with the practice of the method may be made and are contemplated here. hybridization the droplet [0106] All references to bar codes should be understood to include either the bar code or the complement of the bar code, as will be clear from context, and reference to “bar code” or “bar code complement” should be so understood. Likewise, it will be recognized the references to oligonucleotides and segments therein should be understood to include the complement when it is clear from the description that such complementarity with an element is required for the association of bar codes and other elements as described herein.

Orthogonal assays: The methods described herein can be combined with simultaneous profiling of additional modalities such as transcripts and accessible chromatin or tracking of experimental perturbations such as genome edits or extracellular stimuli. See, for example, Peterson et al., 2017, Multiplexed quantification of proteins and transcripts in single cells Nature Biotechnology 35:936-939; Stoeckius et al., 2017, Simultaneous epitope and transcriptome measurement in single cells. Nature Methods 14: 865-868 and Datlinger et al., 2019, Ultra-high throughput single-cell RNA sequencing by combinatorial fluidic indexing. bioRxiv

In an additional embodiment the sequence of the Handle sequence(s) associated with each stained cell is determined. In some embodiments, the Handle is positioned so that it flanked by primer binding sites in the Sequence Fragment Structure, for example, as shown in FIG. 1 (lower panel). In some embodiments the Handle sequence is used in the combinatorial indexing and the deconvolution/demultiplexing process. In some embodiments the Handle sequence is used in the combinatorial indexing and the deconvolution/demultiplexing process and the Pool Oligonucleotide does not include a separate Antibody Barcode Complement sequence and the Handle (or a subsequence within the Handle) has the role of Antibody Barcode.

### 23. Methods

a. Closed Form Derivation of Collision and Empty Droplet Rates

Suppose there are P pools of cells. For pool p, cells arrive according to a Poisson point process with rate λₚ>0 (abbreviated PPP(λₚ)), where the unit of time corresponds to the inter-arrival time of droplets. In the most general formulation, we assume that the point processes for different pools are independent. Further, we assume the probabilities of a gel/bead and a cell encapsulated into a droplet as ρₚ^{b} and ρₚ^{c}, respectively. Therefore, by Poisson thinning, the arrival of cells follows PPP(ρₚ^{c}λₚ).

We are interested in the probability of the event (called collision) that a droplet contains two or more cells from the same pool. Let N, denote the number of cells from pool p successfully loaded into a droplet. Then, N₁, N₂, . . . , Nₚ where Nₚ˜Poisson (ρₚ^{c}λₚ), are independent random variables, and [symbol] [Collision] can be computed as 1− [symbol] [No Droplet Collision]. Here [symbol] [No Droplet Collision] represents a probability that every droplet contains≤1 pool barcode. Therefore, we derive:

$$[ Droplet Collision ] = 1 - [ No Droplet Collision ] = 1 - [ ( {N}_{1} \leq 1 ) ⋂ ( {N}_{2} \leq 1 ) ⋂ … ⋂ ( {N}_{P} \leq 1 ) ] = 1 - [ ( {N}_{1} \leq 1 ) ] [ ( {N}_{2} \leq 1 ) ] … [ ( {N}_{P} \leq 1 ) ] = 1 - {\prod }_{p = 1}^{P} [ {e}^{- {ρ}_{p}^{c} {λ}_{p}} ( 1 + {ρ}_{p}^{c} {λ}_{p} ) ]$$

where the third equality follows from independence.

Next we condition [symbol] [Droplet Collision] on [symbol] [Non-empty Droplet], which is the probability that a droplet contains a cell at a given observation, [symbol] [Non-empty Droplet]=1− [symbol] [Empty Droplet], where:

$$[ Empty Droplet ] = [ ( {N}_{1} = 0 ) ⋂ ( {N}_{2} = 0 ) ⋂ … ⋂ ( {N}_{P} = 0 ) ] = {\prod }_{p = 1}^{P} {e}^{- {ρ}_{p}^{c} {λ}_{p}}$$

If there are D droplets formed and a total of C cells loaded evenly across the P pools (i.e., there are

$$\frac{C}{P}$$

cells per pool), then

$${λ}_{p} = \frac{C {ρ}_{p}^{b}}{PD {ρ}_{p}^{b}} = \frac{C}{PD}$$

for all pools p=1, 2, . . . , P and that ρₚ^{b} becomes a nuisance parameter. If we further assume that ρₚ^{c}=ρ^{c}=1 for all p=1, 2, . . . , P, then [symbol] [Droplet Collision] and [symbol] [Empty Droplet] simplify as

$$[ Droplet Collision ] = 1 - {{e}^{- \frac{C}{D}} [ 1 + \frac{C}{PD} ]}^{P} \\ [ Empty Droplet ] = {e}^{- \frac{C}{D}} .$$

And finally, to estimated conditioned probability of barcode collisions:

$$[ Droplet Collision \vert Non - empty Droplet ] = \frac{[ Droplet Collision ]}{1 - [ Empty Droplet ]} = \frac{1 - {{e}^{- \frac{C}{D}} [ 1 + \frac{C}{PD} ]}^{P}}{1 - {e}^{- \frac{C}{D}}}$$

A second collision rate we can calculate is the cell barcoding (droplet barcode+pool barcode) collision rate which can be computed as the conditional probability that a particular pool p∈{1, 2, . . . , P} has a collision in a given droplet, given that the droplet contains at least one cell from that pool. If we assume that there are D droplets formed and a total of C cells are distributed evenly across P pools, then we obtain:

$$[ Collision in pool p \vert Droplet contains at least one cell from pool p ] = \frac{1 - {e}^{- \frac{C}{PD}} [ 1 + \frac{C}{PD} ]}{1 - {e}^{- \frac{C}{PD}}} ,$$

for all p∈{1, 2, . . . , P}.
The above conditional probability is related to the proportion of the number of pools with a collision in a given droplet, relative to the total number of pools each with at least one cell represented in the droplet. More precisely,

$$\frac{[ Number of pools with a collision in a droplet ]}{[ Number of pools represented at least once in a droplet ]} = \frac{P [ 1 - {e}^{- \frac{C}{PD}} ( 1 + \frac{C}{PD} )}{P [ 1 - {e}^{- \frac{C}{PD}} ]} = \frac{1 - {e}^{- \frac{C}{PD}} [ 1 + \frac{C}{PD} ]}{1 - {e}^{- \frac{C}{PD}}}$$

b. Simulation of Collision and Empty Droplet Rate.

For simulating the collision rates and empty droplet rates, we assumed a cell recovery rate of 60% and 10⁵ droplets are formed per microfluidic reaction resulting in D=6*10⁴. For C cells loaded, cell containing droplets are simulated using a Poisson process where λ=C/D. Assuming each simulated droplet i contains γᵢ cells, we then compute the number of pool barcodes not tagging a cell in each droplet as:

$$BC {0}_{i} = {P ( 1 - \frac{1}{P} )}^{{γ}_{i}}$$

the number of pool barcodes tagging exactly one cell as:

$$BC {1}_{i} = {{C}_{i} ( 1 - \frac{1}{P} )}^{{γ}_{i} - 1}$$

and the number of pool barcodes tagging greater than one cell as:

BCNᵢ=P−BC0ᵢ−BC1ᵢ

The conditional collision rate is estimated as:

[symbol]

[Collision in pool p|Droplet contains at least one cell from pool

$$p ] = \frac{{\sum }_{i}^{C} {BCN}_{i}}{{\sum }_{i}^{C} {BCN}_{i} + {\sum }_{i}^{C} BC {1}_{i}}$$

c. Estimates of Antibody Conjugation, Library Construction, and Sequencing

Cost for library conjugation is estimated to be $4 per antibody per μg using the Thunderlink conjugation kit and assuming averaged costs for input antibodies as purchased for our 60-plex panel. Cost for library preparation is estimated to be $1,500 per well as advertised by 10X Genomics. Cost for sequencing is estimated as $22,484 per 12B reads as advertised by Illumina.

d. Primary Antibody Oligonucleotide Conjugation

For the species mixing experiment, anti-human CD29 and anti-mouse CD29 antibodies were purchased from Biolegend (cat. 303021, 102235) and conjugated per antibody using a ThunderLink kit (Expedeon cat. 425-0000) to distinct 20 bp 3′ amine-modified HPLC-purified oligonucleotides (IDT) to serve as hybridization Handles. Antibodies were conjugated at a ratio of 1 antibody to 3 oligonucleotides (oligos). In parallel, oligos similar to current antibody sequencing tags were directly conjugated at the same ratio for comparison. Sequences for the hybridization oligonucleotides and directly conjugated oligos were designed to be compatible with the 10× feature barcoding system by introducing a reverse complementary sequence to the bead capture sequence, alongside a batch and antibody specific barcode for demultiplexing. Conjugates were quantified using Protein Qubit (Fisher cat. Q33211) for antibody titration and flow validation. Also, we orthogonally quantified using the protein BCA assay. For the human donor mixing experiment, CD4 and CD20 antibodies (Biolegend cat. 300541, 302343) were conjugated as described above.

e. Antibody-Specific Hybridization Design

After conjugation of primary Handle oligos, antibodies were combined and pools of oligos were used to hybridize the primary Handle sequences prior to staining. Of note, only one conjugation was done per antibody with the previously mentioned 20 bp oligonucleotide.

To avoid non-specific transfer of oligonucleotides between the different antibody clones and the same antibody clone from different wells, each clone received a unique 20 bp Handle (Antibody Handle). To sequence with antibody and batch specificity, a 10 bp barcode was added to the Pool Oligo which consisted of a reverse complementary sequence to the antibody specific primary Handle sequence (20 bp), TruSeq Read2 (34 bp), batch barcode (10 bp), and capture sequence (22 bp) (FIG. 2b). Prior to cell staining, 1 ug of each antibody was pooled and hybridized with 1 ul of respective Pool Oligonucleotides at 1 uM at room temperature for 15 minutes. The hybridized antibody-oligonucleotide conjugates were purified using an Amicon 50K MWCO column (Millipore cat. UFC505096) according to the manufacturer's instructions to remove excess free oligonucleotides.

f. Determination of Non-Specific Transfer of Oligonucleotides Between Antibodies

To determine the optimal concentration of hybridizing oligonucleotides for cell staining, we performed a mixed cell line experiment to determine the level of background staining of free oligonucleotides. A mixture of lymphoblastoid cells and primary monocytes were stained with CD14 and CD20 antibodies and hybridized with oligonucleotides with different fluorophores (FAM and Cy5 respectively) per antibody for 15 minutes at room temperature. Concentrations of hybridizing oligonucleotides with different concentrations (1 uM and 100 uM) were tested. Antibodies directly conjugated to fluorophores served as a positive control antibodies (CD13-BV421, Biolegend cat. 562596) to gate respective populations.

g. Validation of Saturation of Hybridization Oligonucleotides Using Flow Cytometry

To determine the saturation of available primary oligo Handles, 1 ug of conjugated CD3 antibody (Biolegend) was hybridized with a 1 ul of 1 uM of a reverse complementary oligo with a Cy5 modification (IDT modification/5Cy5/). After a 15 minute incubation at room temperature, 1 ul of 1 uM of the same reverse complementary oligo but with a FAM modification (IDT modification/56-FAM/) was added to the reaction and additionally incubated for 15 minutes. The cocktail was then added to 1×10⁶ PBMCs pre-stained with Trustain FcX (Biolegend cat. 422302).

h. 10× Genomics Run for SCITO-Seq

Washed and filtered cells were loaded into 10× Genomics V3 Single-Cell 3′ Feature Barcoding technology for Cell Surface Proteins workflow and processed according to the manufacturer's protocol. Afterindex PCR and final elution, all samples were run on the Agilent TapeStation High Sensitivity DNA chip (D5000, Agilent Technologies) to confirm the desired product size. A Qubit 3.0 dsDNA HS assay (ThermoFisherScientific) was used to quantify final library for sequencing. Libraries were sequenced on a NovaSeq 6000 (Read1 28 cycles, index 8 cycles and Read2 98 cycles). R2 cycle can be reduced further for cost reduction (depending on the number of pool+antibody barcode length).

i. Mixed Species Experiment

HeLa and 4T1 cells were ordered from ATCC (ATCC cat. CCL-2, CRL-2539) and cultured in complete DMEM (Fisher cat. 10566016, 10% FBS (Fisher cat. 10083147) and 1% penicillin-streptomycin (Fisher cat. 15140122)) in a 37° C. incubator with 5% CO2 on 10 cm culture dishes (Corning). Prior to staining, cells were trypsinized at 37° C. for 5 minutes using 1 ml Trypsin-EDTA (Fisher cat. 25200056) and were quenched with 10 ml complete DMEM. Cells were harvested and centrifuged at 300×g for 5 minutes. Cells were resuspended in staining buffer (0.01% Tween-20, 2% BSA in PBS) and counted for concentration and viability using a Countess II (Fisher cat. AMQAX1000). HeLa and 4T1 cells were then mixed at equally and 1×10⁶ cells were aliquoted into two 5 ml FACS tubes (Falcon cat. 352052) and volume normalized to 85 ul. Cells were stained with 5 ul of Trustain FcX for 10 minutes on ice. Cell mixtures were stained with a pool of human and mouse CD29 antibodies, either with the direct or universal design, in a total of 100 ul for 45 minutes on ice. Cells were then washed 3 times with 2 ml staining buffer and centrifuged at 300×g for 5 minutes to aspirate supernatant. Cells were then resuspended in 200 ul of staining buffer and counted for concentration and viability as before. Cells from each stained pooled were mixed and 2×10⁴ or 1×10⁵ cells were loaded into the lox chromium controller using 3′ v3 chemistry.

j. Human Donor Mixing Experiment

PBMCs were collected from anonymized healthy donors and were isolated from apheresis residuals by Ficoll gradient. Cells were frozen in 10% DMSO in FBS and stored in a freezing container at −80° C. for one day before long term storage in liquid nitrogen. Cells from two donors were quickly thawed in a 37° C. water bath before being slowly diluted with complete RPMI1640 (Fisher cat. 61870-036, supplemented with 10% FBS and 1% pen-strep) before centrifugation at 300×g for 5 minutes at room temperature. Cells were resuspended in EasySep Buffer (STEMCELL cat. 20144) at a concentration of 5×10⁷ cells/ml before being subject to CD4 and CD20 negative isolation (STEMCELL cat. 17952, 17954). Isolated cells were counted and mixed at a ratio of 3 CD4:1 CD20 for donor 1 and a ratio of 1 CD4:3 CD20 for donor 2 for a total of 1.2×10⁶ cells per donor. The cells were centrifuged at 300×g for 5 minutes at room temperature and resuspended in 85 ul of staining buffer and incubated with 5 ul of Human TruStain FcX (Biolegend cat: 422301) for 10 minutes on ice in 5 ml FACS tubes. Cells from each donor were either mixed prior or stained with well specific barcode hybridized antibody oligo conjugates for 30 minutes on ice. Staining was quenched with the addition of 2 ml staining buffer and washed as previously mentioned. Cells were resuspended in 0.04% BSA in PBS and cells from each well were counted, pooled equally, and then passed through a 40 um strainer (Scienceware cat. H13680-0040). The final strained pool was counted once more prior to loading into a 10× chip B with 2×10⁴ cells, 5×10⁴ cells, 1×10⁵ cells, and 2×10⁵ cells.

k. Mass Cytometry of Healthy Controls

PBMCs were isolated, cryopreserved, and thawed from the same donors as previously described. Once thawed, the cells were counted, and 2×10⁶ cells from each donor were aliquoted into cluster tubes (Corning cat. CLS4401-960EA), and live/dead stained with cisplatin (Sigma cat. P4394) at a final concentration of 5 uM for 5 minutes at room temperature. The live/dead stain was quenched and washed with autoMACS Running Buffer (Miltenyi Biotec cat. 130-091-221). Cells were then stained with 5 uL of TruStain FcX for 10 minutes on ice before surface staining. Mass cytometry antibodies were previously titrated using biological controls to achieve optimal signal to noise ratios. The antibodies in the panel were pooled into a master cocktail and incubated with cells from the two donors and stained for 30 minutes at 4° C. After washing twice with 1 ml autoMACS Running Buffer, the cells were resuspended and fixed in 1.6% PFA (EMS cat. 15710) in MaxPar PBS (Fluidigm cat. 201058) for 10 minutes at room temperature with gentle agitation on an orbital shaker. Samples were then washed twice in autoMACs Running Buffer, and then three times with 1X MaxPar Barcode Perm Buffer (Fluidigm cat. 201057). Each sample was then stained with a unique combination of three purified Palladium isotopes obtained from Matthew Spitzer and the UCSF Flow Cytometry Core for 20 minutes at room temperature with agitation as previously described²⁸. After three washes with autoMACS Running Buffer, samples were combined into one tube and stained with a dilution of 500 uM Cell-ID Intercalator (Fluidigm cat. 201057), to a final concentration of 300 nM in 1.6% PFA in MaxPar PBS at 4° C. until data collection on the CyTOF three days later. Immediately before running on the CyTOF machine, the sample tube was washed once with each autoMACS Running Buffer, MaxPar PBS, and MilliQ H2O. Once all excess proteins and salts were washed out, the sample was diluted in Four Element EQ Calibration Beads (Fluidigm cat. 201078) and MilliQ H2O to a concentration of 1e6 cells/mL and run on a CyTOF Helios at the UCSF Flow Cytometry Core.

I. Comparing Mass Cytometry (CyTOF) and SCITO-Seq

Data was transferred from the CyTOF computer, normalized and de-barcoded using the premessa package (www.github.com/ParkerICI/premessa). Clean files were uploaded to Cytobank (www.ucsf.cytobank.org/) for gating and manual identification of immune cell subsets. Files containing only singlet events were exported from Cytobank and analyzed with CyTOFKit2 package (github.com/JinmiaoChenLab/cytofkit2). Through CyTOFkit2, events were clustered using Rphenograph with k=150 and visualized via UMAP for proportion determination.

m. Pre-Processing and Initial Filtering

Both the species mixing experiments and human donor mixing experiments were processed using Cell Ranger 3.0 Feature Barcoding Analysis using default parameters. For cDNA and ADT alignment, we specified the input library type as ‘Gene Expression’ and ‘Antibody Capture’ respectively as recommended. For ADT alignment, specific barcode sequences (Ab+pool) were specified as a reference. Reads were aligned to the hg19 and mm10 concatenation reference for species mixing experiment. For all human experiments, the reads were aligned to the human reference genome (GRCh38/hg20). We first removed RBC and Platelets and removed cells with more than 15% of mitochondrial gene related reads. We further removed genes with less than 1 counts across all cells.

n. Normalization for Species Mixing and T/B Cell Human Donor Mixing Experiment

For cDNA counts, data was normalized by dividing each UMI counts to the total UMI counts and multiplied by 10,000. Then, the data was log 1p transformed (numpy.log 1p). Finally, the data was scaled to have mean=0 and standard deviation=1. Clustering was done using the Leiden algorithm²⁹ using 10 nearest neighbors and a resolution of 0.2 for mixed species and two-donor experiment with two cell types (T and B cells).

To normalize ADT counts in species mixing experiment, the data was log transformed and standardized to have mean=0 and standard deviation=1. For ADT counts in two human donor mixing experiment with two cell types, after log transformation of the raw data, we used a Gaussian Mixture Model in scikit-learn package in python to normalize the data with the following parameters (convergence threshold 1e-3 and max iteration to 100, number of components 2). The data was normalized by z-score like transformation (log transformed raw value−mean of the posterior means of two components/mean of the posterior standard deviations).

o. Implementation of an Algorithm for Batch Demultiplexing and Multiplet Resolution

Considering all antibodies in each pool, we normalized each value by dividing mean expression value of CD45 counts across all pool (considered as a universal expression marker) for each droplet barcode yielding a p*m matrix (p is the number of pool and m is number of droplet barcodes). Then, the matrix was CLR normalized and demultiplexed using HTODemux from Seurat (v3.0) (www.satijalab.org/seurat/) to classify the droplet barcode to a pool or unassigned (we discretized the value of 0 or 1). Using this binary matrix, we iterated over p times (where discretized value equals 1) to get final resolved matrix of (n*r) where n is the number of antibodies used and r is the resolved number of cells. For each iteration, we selected the columns that were positive for the above-mentioned discretized matrix. An additional round of HTODemux was used to re-classify the ‘Negative’ cells from initial classification because most of the initial classification which deemed the cells negative had a UMAP distributions which were contained in the original clusters.

p. Analysis of PBMC Experiment: Normalization and Resolution of Multiplets

To normalize cDNA data for PBMC experiments, we used the same normalization method as described above. To generate UMAP based on ADT counts for PBMC experiment, we performed batch demultiplexing the multiplet resolution using the algorithm described previously. Then, the resolved matrix (n*r) goes through similar normalization as in the cDNA processing. Raw values are normalized to total counts of 10,000 per cell and log 1p transformed. Then, the values are standardized (mean 0, standard deviation 1) per batch. Using this normalized values, PCA was performed to reduce the dimensionality. Leiden clustering was done with 10 neighbors and 15 PCs from the previous step. Resolution value for 1.0 is used to assign clusters for whole PBMC experiments. Finally, UMAP was run to visualize resolved total cells. To remove collided cells in 60-plex and 165-plex experiment, we computed the average number of UMIs expressed per cell and thresholded cells based on the quantile distribution (>80% in the UMI distribution is filtered out) to remove cells and also manually inspect expression across all leiden clusters to exclude the cluster that expresses multiple markers.

q. Analysis of PBMC Experiment: Demultiplexing Donor Identity

For demultiplexing the donors, a VCF file containing donor genotype information and the bam file output from the Cell Ranger pipeline were used as inputs for demuxlet (Freemuxlet) with default parameters. For donors without genotypic information, we used Freemuxlet (https://github.com/statgen/popscle/) to assign droplet barcodes to the corresponding donor.

r. Analysis of PBMC Experiment: Downsampling Experiment with Adjusted Rand Index Calculations

To evaluate the quality of clustering at a given downsample, Adjusted Rand Index (ARI) was used as the comparison metric. Leiden clustering was performed on the full dataset and resulting cluster labels were taken as ground truth cell type assignments. To determine an optimal Leiden resolution for downsampling, clustering was performed 5 times at a range of resolutions. A resolution that produced consistently high ARI was then used to generate ground truth labels and perform clustering on downsampled data. Data was downsampled to a specified mean UMI/Antibody/cell using scanpy (1.4.5.post3) to downsample total reads. Downsampled data was then clustered and labels compared to full dataset clustering with ARI.

### 24. REFERENCES

1. Macosko, E. Z. et al. Highly Parallel Genome-wide Expression Profiling of Individual Cells Using Nanoliter Droplets. Cell 161, 1202-1214 (2015).
2. Klein, A. M. et al. Droplet barcoding for single-cell transcriptomics applied to embryonic stem cells. Cell 161, 1187-1201 (2015).
3. Buenrostro, J. D. et al. Single-cell chromatin accessibility reveals principles of regulatory variation. Nature 523, 486-490 (2015).
4. Stoeckius, M. et al. Simultaneous epitope and transcriptome measurement in single cells. Nat. Methods 14, 865-868 (2017).
5. Shahi, P., Kim, S. C., Haliburton, J. R., Gartner, Z. J. & Abate, A. R. Abseq: Ultrahigh-throughput single cell protein profiling with droplet microfluidic barcoding. Sci. Rep. 7, 44447 (2017).
6. Gerlach, J. P. et al. Combined quantification of intracellular (phospho-)proteins and transcriptomics from fixed single cells. doi:10.1101/356329.
7. Peterson, V. M. et al. Multiplexed quantification of proteins and transcripts in single cells. Nat. Biotechnol. 35, 936-939 (2017).
8. Bandura, D. R. et al. Mass cytometry: technique for real time single cell multitarget immunoassay based on inductively coupled plasma time-of-flight mass spectrometry. Anal. Chem. 81, 6813-6822 (2009).
9. Spitzer, M. H. & Nolan, G. P. Mass Cytometry: Single Cells, Many Features. Cell 165, 780-791 (2016).
10. Kang, H. M. et al. Multiplexed droplet single-cell RNA-sequencing using natural genetic variation. Nat. Biotechnol. 36, 89-94 (2018).
11. McGinnis, C. S. et al. MULTI-seq: sample multiplexing for single-cell RNA sequencing using lipid-tagged indices. Nature Methods vol. 16 619-626 (2019).
12. Stoeckius, M. et al. Cell Hashing with barcoded antibodies enables multiplexing and doublet detection for single cell genomics. Genome Biol. 19, 224 (2018).
13. Datlinger, P. et al. Pooled CRISPR screening with single-cell transcriptome readout. Nat. Methods 14, 297-301 (2017).
14. Mimitou, E. P. et al. Multiplexed detection of proteins, transcriptomes, clonotypes and CRISPR perturbations in single cells. Nat. Methods 16, 409-412 (2019).
15. Marguerat, S. et al. Quantitative analysis of fission yeast transcriptomes and proteomes in proliferating and quiescent cells. Cell 151, 671-683 (2012).
16. Zheng, G. X. Y. et al. Massively parallel digital transcriptional profiling of single cells. Nat. Commun. 8, 14049 (2017).
17. Cao, J. et al. Comprehensive single-cell transcriptional profiling of a multicellular organism. Science vol. 357 661-667 (2017).
18. Cao, J. et al. Joint profiling of chromatin accessibility and gene expression in thousands of single cells. Science 361, 1380-1385 (2018).
19. Cao, J. et al. The single-cell transcriptional landscape of mammalian organogenesis. Nature 566, 496-502 (2019).
20. Rosenberg, A. B. et al. Single-cell profiling of the developing mouse brain and spinal cord with split-pool barcoding. Science 360, 176-182 (2018).
21. Lareau, C. A. et al. Droplet-based combinatorial indexing for massive-scale single-cell chromatin accessibility. Nature Biotechnology vol. 37 916-924 (2019).
22. Datlinger, P., Rendeiro, A. F., Boenke, T., Krausgruber, T., Barreca, D., Bock, C., Ultra-high throughput single-cell RNA sequencing by combinatorial fluidic indexing. bioRxiv (2019) 12.17.879304; doi: https://doi.org/10.1101/2019.12.17.879304
23. Huang, Y., McCarthy, D. J. & Stegle, O. Vireo: Bayesian demultiplexing of pooled single-cell RNA-seq data without genotype reference. Genome Biol. 20, 273 (2019).
24. Heaton, H. et al. souporcell: Robust clustering of single cell RNAseq by genotype and ambient RNA inference without reference genotypes. bioRxiv 699637 (2019) doi:10.1101/699637.
25. Gehring, J., Hwee Park, J., Chen, S., Thomson, M. & Pachter, L. Highly multiplexed single-cell RNA-seq by DNA oligonucleotide tagging of cellular proteins. Nat. Biotechnol. 38, 35-38 (2020).
26. Ferrer-Font, L. et al. Panel Design and Optimization for High-Dimensional Immunophenotyping Assays Using Spectral Flow Cytometry. Current Protocols in Cytometry 92 (2020).
27. Collin, M et al. Human dendritic cell subsets: an update. Immunology 154, 3-20 (2018).
28. Zunder, E. R. et al. Palladium-based mass tag cell barcoding with a doublet-filtering scheme and single-cell deconvolution algorithm. Nat. Protoc. 10, 316-333 (2015).
29. Traag, V. A., Waltman, L. & van Eck, N. J. From Louvain to Leiden: guaranteeing well-connected communities. Sci. Rep. 9, 5233 (2019).

The invention has been described in this disclosure with reference to the specific examples and illustrations. The features of these examples and illustrations do not limit the practice of the claimed invention, unless explicitly stated or otherwise required. Changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

For all purposes in the United States of America, each and every publication and patent document referred to in this disclosure is incorporated herein by reference in its entirety to the same extent as if each such publication or document was specifically and individually indicated to be incorporated herein by reference.

### REFERENCE TO A SEQUENCE LISTING

The Sequence Listing written in file 103182-1233370-004510WO_SL.txt created on Apr. 30, 2021, 1 KB, machine format IBM-PC, MS-Windows operating system, is hereby incorporated by reference in its entirety for all purposes.

1. An assay method comprising
i) tagging cell surface proteins of a population of cells with DNA-barcoded antibodies,
ii) distributing the cells into droplets, wherein least 30% of occupied droplets contain two or more cells,
iii) determining cell surface protein expression profiles for individual cells of the multiply encapsulated cells by resolving a combinatorial index of barcodes.

2. The method of claim 1 further comprising determining cell surface protein expression profiles for the singly encapsulated cells.

3. The method of claim 1 wherein at least 30% of occupied droplets, optionally at least 50% of occupied droplets, comprise two cells.

4. The method of claim 1 wherein the combinatorial index of barcodes comprises an antibody barcode, a pool barcode and a droplet barcode; and/or the combinatorial index of barcodes further comprises a UMI.

5. (canceled)

6. An assay method for determining cell surface protein expression profiles of cells in a population of cells, comprising
i) dividing the population of cells into a plurality of subpopulations of cells;
ii) tagging the cell surface proteins of cells in each subpopulation, wherein the tagging comprises combining the subpopulation with a plurality or panel of handle-tagged antibodies (HTAs), wherein each HTA binds a specified cell surface protein of interest, each HTA is associated with or becomes associated with an antibody barcode, and each HTA is, or becomes, associated with a pool barcode identifying the subpopulation; thereby producing stained cells;
iii) distributing the stained cells to compartments such as droplets,
wherein, of the compartments that are occupied (contain cells) at least 30% contain 2 or more cells,
or
wherein, the compartments are loaded according to a Poisson distribution in which lambda is greater than 1, optionally greater than 2, optionally greater than 3
wherein each compartment is identified by a compartment-specific barcode, and wherein the compartment-specific barcode becomes associated with an antibody barcode and its associated pool barcode;
iv) producing a plurality of polynucleotides, each polynucleotide comprising a combination of a compartment-specific barcode, an antibody barcode and a pool barcode, wherein said barcodes were associated with each other in step (iii);
iv) determining the combinations of barcodes produced in iv.

7. The method of claim 6 wherein after step (ii) and before step (iii) the stained cells are fixed and permeabilized.

8. The method of claim 6 wherein the compartments in step (iii) are droplets.

9. The method of claim 6 wherein the polynucleotides produced in step (iv) are produced by transcription or amplification.

10. The method of claim 6 wherein the polynucleotides produced in step (iv) are sequenced, thereby determining the combinations of a compartment-specific barcode, an antibody barcode, a pool barcode, and optionally a UMI, produced in step (iii).

11. The method of claim 6 wherein in step (ii), HTA and pool barcodes are associated by formation of a nucleic acid duplex, or pool barcodes and droplet barcodes are associated by formation of a HTA and pool barcodes are associated by formation of a nucleic acid duplex, or pool barcodes and droplet barcodes are associated by ligation.

12-13. (canceled)

14. The method of claim 11 wherein pool barcodes and droplet barcodes are associated by ligation, and the Pool Oligonucleotide has a ligatable (e.g., phosphorylated) 5′ terminus that is ligated to the 3′-terminus of the Droplet Oligonucleotide.

15. The method of claim 14 where the ligation is carried out in the presence of a bridge oligonucleotide that links the Pool Oligonucleotide and the Droplet Oligonucleotide.

16. An assay method comprising
(a) providing a plurality of vessels, each vessel comprising
i-a) a plurality of cells from a population, each cell comprising a plurality of cell surface proteins, and
ii-a) a panel of staining constructs, wherein each staining construct comprises a handle-tagged antibody and a pool oligonucleotide,
wherein each handle-tagged antibody comprises
iii-a) an antibody specific for a cell surface protein in (i-a), and
iv-a) a handle oligonucleotide attached to the antibody,
wherein the handle oligonucleotide comprises a handle sequence that identifies the specificity of the antibody to which it is attached; and
each pool oligonucleotide comprises the following nucleotide segments:
v-a) a handle complement segment complementary to, and annealed to, the handle oligonucleotide,
vi-a) a capture complement segment,
vii-a) an antibody barcode complement segment having a sequence that identifies the binding specificity of the antibody in (iii-a) and thereby identifies the handle oligonucleotide in (iv-a),
viii-a) a pool barcode complement segment,
wherein (vii-a) and (viii-a) are positioned between (v-a) and (vi-a),
wherein in each vessel, the staining constructs in the vessel have the same pool barcode complement segments,
wherein in at least some vessels at least one staining construct is to a cell surface protein in i-a);
(b) optionally combining the contents of all or some of said plurality of vessels,
(c) loading individual stained cells or combinations of individual stained cells into compartments,
wherein each stained cell comprises one or more staining constructs bound to a cell surface protein of the cell
wherein at least some compartments comprise one or more stained cells and a plurality of droplet oligonucleotides
wherein each droplet oligonucleotide comprises a droplet bar code and a capture segment
wherein the droplet oligonucleotides in a compartment have the same droplet barcode and droplet oligonucleotides in different compartments have different barcodes
wherein the capture segment is complementary to and anneals to the capture complement segment of the pool oligonucleotide;
(d) producing sequence fragment structures corresponding to the capture constructs, each sequence fragment structure comprising a droplet barcode, a pool barcode and an antibody barcode whereby a plurality of sequence fragment structures are produced;
(e) sequencing at least some of the plurality of sequence fragment structures to determine the sequences of the droplet barcode, the pool barcode and the antibody barcode of individual sequence fragment structures;
(f) determining from the sequencing in (e) distribution of cell surface proteins on individual cells.

17. An assay method comprising carrying out the method of claim 16, except that the capture segment of the droplet oligonucleotide is ligated to the capture segment (complement of capture complement) of the pool oligonucleotide rather than associated by hybridization, wherein optionally the ligation is carried out in the presence of a bridge oligonucleotide that links the Pool Oligonucleotide and the Droplet Oligonucleotide.

18. The method of claim 16 wherein the cells in the plurality of vessels in (a) comprise a cell population and a composition or expression of cell surface proteins in the population is determined; or wherein the compartments are droplets or wells: or wherein the droplet oligonucleotides are attached to beads.

19-20. (canceled)

21. The method of claim 16 wherein in step (c) at least some of the compartments have two or more cells loaded therein, and cell surface protein expression profiles of said two or more cells are determined.

22. The method of claim 21 wherein at least 50% of the compartments containing cells comprise two or more cells.

23. The method of claim 16 wherein the pool barcode and antibody barcode are a compound barcode.

24. A kit comprising two or more of
i) a plurality of handle-tagged antibodies comprising different handle sequences and antibodies with different binding specificities, wherein there is a correlation between each handle sequence and each antibody specificity;
ii) a plurality of pool oligonucleotides with different handle complement sequences, wherein said handle complement sequences are complementary to and can anneal to the handle sequences in (i);
iii) a plurality of droplet oligonucleotides configured to combine with pool oligonucleotides.

25. The kit of claim 24 comprising (i), (ii) and (iii).

26-27. (canceled)
